SEQUENCE LISTING (21 sequences)

SEQ ID NO: 1 - protein, 429 aa, Podocoryna carnea
GPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLRGKPGAKGIAGSDGEAGESGAPGQSGPTGPRGQRGPSGEDGNPGLQGLPGSDGEPGEEGQPGRSGQPGQQGPRGSPGEVGPRGSKGPSGDRGDRGERGVPGQTGSAGNVGEDGEQGGKGVDGASGPSGALGARGPPGSRGDTGAVGPPGPTGRSGLPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 2 - DNA, 1289 bp, Podocoryna carnea
ggaccacaaggtgttgtaggagctgatggcaaagatggaacaccgggagagaaaggtgagcaaggacgaaccggagctgcaggaaaacagggaagccctggagcagatggagcaagaggccctcttggatcaattggacaacaaggtgctcgtggagaacctggtgatccaggatctcccggcttaagaggagatactggattggctggagtcaaaggagtagcaggaccatctggtcgacctggacaacccggtgcaaatggattacctggtgtgaatggcagaggcggtttgagaggcaaacctggtgctaaaggaattgctggcagtgatggagaagcgggagaatctggcgcacctggacagtccggacctaccggtccacgtggtcaacgaggaccaagtggtgaggatggtaatcctggattacagggattgcctggttctgatggagagcccggagaggaaggacaacctggaagatctggtcaaccaggacagcaaggaccacgtggttcccctggagaggtaggaccaagaggatctaaaggtccatcaggagatcgtggtgacaggggagagagaggtgttcctggacaaacaggttcggctggaaatgtaggagaagatggagagcaaggaggcaaaggtgtcgatggagcgagtggaccaagtggagctcttggtgctcgtggtcccccaggaagtagaggtgacaccggggcagtgggacctcccggacctactgggcgatctggtttacctggaaacgcaggacaaaagggaccaagtggtgaaccaggtagtccaggaaaagcaggatcagctggtgaacagggtcctcctggtaaagacggatcaaatggtgaacctggatctcctggcaaagagggtgaacgtggtcttgctggtccaccaggtccagatggcagacgtggtgaaacgggatctccaggtatcgctggtgctcttggtaaaccaggtttggaaggacctaaaggttatccaggattaagaggaagagatggaaccaatggcaaacgaggagaacaaggagaaactggtcctgatggagtcagaggtattcctggaaatgatggacaatctggcaaaccaggtattgatggtattgacggaacaaatggtcaaccaggtgaggctggataccaaggtggtagaggtacacgtggtcagttaggtgaaactggtgatgtcggacagaatggagatcgaggagctcctggtcctgatggatctaaaggttctgctggtagaccaggacttcgtgg

SEQ ID NO: 3 - DNA, 1425 bp, Artificial Sequence
  Description of Artificial Sequence Synthetic polynucleotide
atgaaaaagatttggctggcgctggctggtttagttttagcgtttagcgcatcggcggcgcagtatgaagatcaccatcaccaccaccaccatcaccactctggctcgagcctggtgccgcgcggcagccatatgggtccgcagggtgttgttggtgcagatggtaaagacggtaccccgggtgaaaaaggagaacagggacgtacaggtgcagcaggtaaacagggcagcccgggtgccgatggtgcccgtggcccgctgggtagcattggtcagcagggtgcaagaggcgaaccgggcgatccgggtagtccgggcctgcgtggtgatacgggtctggccggtgttaaaggcgttgcaggtccttcaggtcgtccaggtcaaccgggtgcaaatggtctgccgggtgttaatggtcgtggcggtctgcgtggcaaaccgggagcaaaaggtattgcaggtagcgatggagaagccggtgaaagcggtgccccgggtcagagtggtccgaccggtccgcgcggtcagcgtggtccgtctggtgaagatggcaatccgggtctgcagggtctgcctggtagtgatggcgaaccaggtgaagaaggtcagccgggtcgttcaggccagccgggccagcagggcccgcgtggtagcccgggcgaagttggcccgcggggtagtaaaggtcctagtggcgatcgcggtgatcgtggtgaacgcggtgttcctggtcagaccggtagcgcaggtaatgttggcgaagatggtgaacagggtggcaaaggtgttgatggtgcaagcggtccgagcggtgcactgggtgcacgtggtcctccgggcagccgtggtgacaccggtgcagttggtccgcctggcccgaccggccgtagtggcttaccgggtaatgcaggtcagaaaggtccgtcaggtgaacctggcagccctggtaaagcaggtagtgccggtgagcagggtccgccgggcaaagatggtagtaatggtgagccgggtagccctggcaaagaaggtgaacgtggtctggcaggaccgccgggtcctgatggtcgccgcggtgaaacgggttcaccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaacaggtgatgttggtcagaatggtgatcgcggcgcaccgggtccggatggtagcaaaggtagcgccggtcgtccgggtttacgttaa

SEQ ID NO: 4 - DNA, 1425 bp, Artificial Sequence
  Description of Artificial Sequence Synthetic polynucleotide
atgaaaaagatttggctggcgctggctggtttagttttagcgtttagcgcatcggcggcgcagtatgaagatcaccatcaccaccaccaccatcaccactctggctcgagcctggtgccgcgcggcagccatatgggtccgcagggtgttgttggtgcagatggtaaagacggtaccccgggtgaaaaaggtgaacagggtcgtaccggtgcagcaggtaaacagggcagcccgggtgccgatggtgcccgtggcccgctgggtagcattggtcagcagggtgcacgtggcgaaccgggcgatccgggtagcccgggcctgcgtggtgatacgggtctggccggtgttaaaggcgttgcaggtccttctggtcgtccaggtcaaccgggtgcaaatggtctgccgggtgttaatggtcgtggcggtctgcgtggcaaaccgggtgcaaaaggtattgcaggtagcgatggcgaagccggtgaaagcggtgccccgggtcagagcggtccgaccggtccgcgcggtcagcgtggtccgtctggtgaagatggcaatccgggtctgcagggtctgcctggtagcgatggcgaaccaggtgaagaaggtcagccgggtcgttctggccagccgggccagcagggcccgcgtggtagcccgggcgaagttggcccgcgcggttctaaaggtcctagcggcgatcgcggtgatcgtggtgaacgcggtgttcctggtcagaccggtagcgcaggtaatgttggcgaagatggtgaacagggtggcaaaggtgttgatggtgcaagcggtccgagcggtgcactgggtgcacgtggtcctccgggcagccgtggtgacaccggtgcagttggtccgcctggcccgaccggccgtagcggcctgccgggtaatgcaggtcagaaaggtccgtctggtgaacctggcagccctggtaaagcaggtagcgccggtgagcagggtccgccgggcaaagatggtagcaatggtgagccgggtagccctggcaaagaaggtgaacgtggtctggcaggtccgccgggtcctgatggtcgccgcggtgaaacgggttctccgggtattgccggtgccctgggtaaaccaggtctggaaggtccgaaaggttatcctggtctgcgcggtcgtgatggtaccaatggcaaacgtggcgaacagggcgaaaccggtccagatggtgttcgtggtattccgggtaacgatggtcagagcggtaaaccgggcattgatggtattgatggcaccaatggtcagcctggcgaagcaggttatcagggtggtcgcggtacccgtggtcagctgggtgaaaccggtgatgttggtcagaatggtgatcgcggcgcaccgggtccggatggtagcaaaggtagcgccggtcgtccgggtctgcgttaa

SEQ ID NO: 5 - protein, 474 aa, Artificial Sequence
  Description of Artificial Sequence Synthetic polypeptide
MKKIWLALAGLVLAFSASAAQYEDHHHHHHHHHSGSSLVPRGSHMGPQGVVGADGKDGTPGEKGEQGRTGAAGKQGSPGADGARGPLGSIGQQGARGEPGDPGSPGLRGDTGLAGVKGVAGPSGRPGQPGANGLPGVNGRGGLRGKPGAKGIAGSDGEAGESGAPGQSGPTGPRGQRGPSGEDGNPGLQGLPGSDGEPGEEGQPGRSGQPGQQGPRGSPGEVGPRGSKGPSGDRGDRGERGVPGQTGSAGNVGEDGEQGGKGVDGASGPSGALGARGPPGSRGDTGAVGPPGPTGRSGLPGNAGQKGPSGEPGSPGKAGSAGEQGPPGKDGSNGEPGSPGKEGERGLAGPPGPDGRRGETGSPGIAGALGKPGLEGPKGYPGLRGRDGTNGKRGEQGETGPDGVRGIPGNDGQSGKPGIDGIDGTNGQPGEAGYQGGRGTRGQLGETGDVGQNGDRGAPGPDGSKGSAGRPGLR

SEQ ID NO: 6 - protein, 30 aa, Artificial Sequence
  Description of Artificial Sequence Synthetic His tag
  MISC_FEATURE (1)..(30): This sequence may encompass 2-30 residues
HHHHHHHHHHHHHHHHHHHHHHHHHHHHHH

SEQ ID NO: 7 - protein, 339 aa, Artificial Sequence
  Description of Artificial Sequence Synthetic polypeptide
  MOD_RES (2)..(2): Hydroxyproline
  MOD_RES (5)..(5): Hydroxyproline
  MOD_RES (8)..(8): Hydroxyproline
  MOD_RES (11)..(11): Hydroxyproline
  MOD_RES (14)..(14): Hydroxyproline
  MOD_RES (17)..(17): Hydroxyproline
  MOD_RES (20)..(20): Hydroxyproline
  MOD_RES (23)..(23): Hydroxyproline
  MOD_RES (26)..(26): Hydroxyproline
  MOD_RES (29)..(29): Hydroxyproline
  MOD_RES (32)..(32): Hydroxyproline
  MOD_RES (35)..(35): Hydroxyproline
  MOD_RES (38)..(38): Hydroxyproline
  MOD_RES (41)..(41): Hydroxyproline
  MOD_RES (44)..(44): Hydroxyproline
  MOD_RES (47)..(47): Hydroxyproline
  MOD_RES (50)..(50): Hydroxyproline
  MOD_RES (53)..(53): Hydroxyproline
  MOD_RES (56)..(56): Hydroxyproline
  MOD_RES (59)..(59): Hydroxyproline
  MOD_RES (62)..(62): Hydroxyproline
  MOD_RES (65)..(65): Hydroxyproline
  MOD_RES (68)..(68): Hydroxyproline
  MOD_RES (71)..(71): Hydroxyproline
  MOD_RES (74)..(74): Hydroxyproline
  MOD_RES (77)..(77): Hydroxyproline
  MOD_RES (80)..(80): Hydroxyproline
  MOD_RES (83)..(83): Hydroxyproline
  MOD_RES (86)..(86): Hydroxyproline
  MOD_RES (89)..(89): Hydroxyproline
  MOD_RES (92)..(92): Hydroxyproline
  MOD_RES (95)..(95): Hydroxyproline
  MOD_RES (98)..(98): Hydroxyproline
  MOD_RES (101)..(101): Hydroxyproline
  MOD_RES (104)..(104): Hydroxyproline
  MOD_RES (107)..(107): Hydroxyproline
  MOD_RES (110)..(110): Hydroxyproline
  MOD_RES (113)..(113): Hydroxyproline
  MOD_RES (116)..(116): Hydroxyproline
  MOD_RES (119)..(119): Hydroxyproline
  MOD_RES (122)..(122): Hydroxyproline
  MOD_RES (125)..(125): Hydroxyproline
  MOD_RES (128)..(128): Hydroxyproline
  MOD_RES (131)..(131): Hydroxyproline
  MOD_RES (134)..(134): Hydroxyproline
  MOD_RES (137)..(137): Hydroxyproline
  MOD_RES (140)..(140): Hydroxyproline
  MOD_RES (143)..(143): Hydroxyproline
  MOD_RES (146)..(146): Hydroxyproline
  MOD_RES (149)..(149): Hydroxyproline
  MOD_RES (152)..(152): Hydroxyproline
  MOD_RES (155)..(155): Hydroxyproline
  MOD_RES (158)..(158): Hydroxyproline
  MOD_RES (161)..(161): Hydroxyproline
  MOD_RES (164)..(164): Hydroxyproline
  MOD_RES (167)..(167): Hydroxyproline
  MOD_RES (170)..(170): Hydroxyproline
  MOD_RES (173)..(173): Hydroxyproline
  MOD_RES (176)..(176): Hydroxyproline
  MOD_RES (179)..(179): Hydroxyproline
  MOD_RES (182)..(182): Hydroxyproline
  MOD_RES (185)..(185): Hydroxyproline
  MOD_RES (188)..(188): Hydroxyproline
  MOD_RES (191)..(191): Hydroxyproline
  MOD_RES (194)..(194): Hydroxyproline
  MOD_RES (197)..(197): Hydroxyproline
  MOD_RES (200)..(200): Hydroxyproline
  MOD_RES (203)..(203): Hydroxyproline
  MOD_RES (206)..(206): Hydroxyproline
  MOD_RES (209)..(209): Hydroxyproline
  MOD_RES (212)..(212): Hydroxyproline
  MOD_RES (215)..(215): Hydroxyproline
  MOD_RES (218)..(218): Hydroxyproline
  MOD_RES (221)..(221): Hydroxyproline
  MOD_RES (224)..(224): Hydroxyproline
  MOD_RES (227)..(227): Hydroxyproline
  MOD_RES (230)..(230): Hydroxyproline
  MOD_RES (233)..(233): Hydroxyproline
  MOD_RES (236)..(236): Hydroxyproline
  MOD_RES (239)..(239): Hydroxyproline
  MOD_RES (242)..(242): Hydroxyproline
  MOD_RES (245)..(245): Hydroxyproline
  MOD_RES (248)..(248): Hydroxyproline
  MOD_RES (251)..(251): Hydroxyproline
  MOD_RES (254)..(254): Hydroxyproline
  MOD_RES (257)..(257): Hydroxyproline
  MOD_RES (260)..(260): Hydroxyproline
  MOD_RES (263)..(263): Hydroxyproline
  MOD_RES (266)..(266): Hydroxyproline
  MOD_RES (269)..(269): Hydroxyproline
  MOD_RES (272)..(272): Hydroxyproline
  MOD_RES (275)..(275): Hydroxyproline
  MOD_RES (278)..(278): Hydroxyproline
  MOD_RES (281)..(281): Hydroxyproline
  MOD_RES (284)..(284): Hydroxyproline
  MOD_RES (287)..(287): Hydroxyproline
  MOD_RES (290)..(290): Hydroxyproline
  MOD_RES (293)..(293): Hydroxyproline
  MOD_RES (296)..(296): Hydroxyproline
  MOD_RES (299)..(299): Hydroxyproline
  MOD_RES (317)..(317): Hydroxyproline
  MOD_RES (320)..(320): Hydroxyproline
  MOD_RES (323)..(323): Hydroxyproline
  MOD_RES (326)..(326): Hydroxyproline
  MOD_RES (329)..(329): Hydroxyproline
  MOD_RES (332)..(332): Hydroxyproline
  MOD_RES (335)..(335): Hydroxyproline
  MOD_RES (338)..(338): Hydroxyproline
PPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPEGPEGPEGPEGPEGPPGPPGPPGPPGPPGPPGPPGPPG

SEQ ID NO: 8 - protein, 300 aa, Artificial Sequence
  Description of Artificial Sequence Synthetic polypeptide
  MOD_RES (2)..(2): Hydroxyproline
  MOD_RES (5)..(5): Hydroxyproline
  MOD_RES (8)..(8): Hydroxyproline
  MOD_RES (11)..(11): Hydroxyproline
  MOD_RES (14)..(14): Hydroxyproline
  MOD_RES (17)..(17): Hydroxyproline
  MOD_RES (20)..(20): Hydroxyproline
  MOD_RES (23)..(23): Hydroxyproline
  MOD_RES (26)..(26): Hydroxyproline
  MOD_RES (29)..(29): Hydroxyproline
  MOD_RES (32)..(32): Hydroxyproline
  MOD_RES (35)..(35): Hydroxyproline
  MOD_RES (38)..(38): Hydroxyproline
  MOD_RES (41)..(41): Hydroxyproline
  MOD_RES (44)..(44): Hydroxyproline
  MOD_RES (47)..(47): Hydroxyproline
  MOD_RES (50)..(50): Hydroxyproline
  MOD_RES (53)..(53): Hydroxyproline
  MOD_RES (56)..(56): Hydroxyproline
  MOD_RES (59)..(59): Hydroxyproline
  MOD_RES (62)..(62): Hydroxyproline
  MOD_RES (65)..(65): Hydroxyproline
  MOD_RES (68)..(68): Hydroxyproline
  MOD_RES (71)..(71): Hydroxyproline
  MOD_RES (74)..(74): Hydroxyproline
  MOD_RES (77)..(77): Hydroxyproline
  MOD_RES (80)..(80): Hydroxyproline
  MOD_RES (83)..(83): Hydroxyproline
  MOD_RES (86)..(86): Hydroxyproline
  MOD_RES (89)..(89): Hydroxyproline
  MOD_RES (92)..(92): Hydroxyproline
  MOD_RES (95)..(95): Hydroxyproline
  MOD_RES (98)..(98): Hydroxyproline
  MOD_RES (101)..(101): Hydroxyproline
  MOD_RES (104)..(104): Hydroxyproline
  MOD_RES (107)..(107): Hydroxyproline
  MOD_RES (110)..(110): Hydroxyproline
  MOD_RES (113)..(113): Hydroxyproline
  MOD_RES (116)..(116): Hydroxyproline
  MOD_RES (119)..(119): Hydroxyproline
  MOD_RES (122)..(122): Hydroxyproline
  MOD_RES (125)..(125): Hydroxyproline
  MOD_RES (128)..(128): Hydroxyproline
  MOD_RES (131)..(131): Hydroxyproline
  MOD_RES (134)..(134): Hydroxyproline
  MOD_RES (137)..(137): Hydroxyproline
  MOD_RES (140)..(140): Hydroxyproline
  MOD_RES (143)..(143): Hydroxyproline
  MOD_RES (146)..(146): Hydroxyproline
  MOD_RES (149)..(149): Hydroxyproline
  MOD_RES (152)..(152): Hydroxyproline
  MOD_RES (155)..(155): Hydroxyproline
  MOD_RES (158)..(158): Hydroxyproline
  MOD_RES (161)..(161): Hydroxyproline
  MOD_RES (164)..(164): Hydroxyproline
  MOD_RES (167)..(167): Hydroxyproline
  MOD_RES (170)..(170): Hydroxyproline
  MOD_RES (173)..(173): Hydroxyproline
  MOD_RES (176)..(176): Hydroxyproline
  MOD_RES (179)..(179): Hydroxyproline
  MOD_RES (182)..(182): Hydroxyproline
  MOD_RES (185)..(185): Hydroxyproline
  MOD_RES (188)..(188): Hydroxyproline
  MOD_RES (191)..(191): Hydroxyproline
  MOD_RES (194)..(194): Hydroxyproline
  MOD_RES (197)..(197): Hydroxyproline
  MOD_RES (200)..(200): Hydroxyproline
  MOD_RES (203)..(203): Hydroxyproline
  MOD_RES (206)..(206): Hydroxyproline
  MOD_RES (209)..(209): Hydroxyproline
  MOD_RES (212)..(212): Hydroxyproline
  MOD_RES (215)..(215): Hydroxyproline
  MOD_RES (218)..(218): Hydroxyproline
  MOD_RES (221)..(221): Hydroxyproline
  MOD_RES (224)..(224): Hydroxyproline
  MOD_RES (227)..(227): Hydroxyproline
  MOD_RES (230)..(230): Hydroxyproline
  MOD_RES (233)..(233): Hydroxyproline
  MOD_RES (236)..(236): Hydroxyproline
  MOD_RES (239)..(239): Hydroxyproline
  MOD_RES (242)..(242): Hydroxyproline
  MOD_RES (245)..(245): Hydroxyproline
  MOD_RES (248)..(248): Hydroxyproline
  MOD_RES (251)..(251): Hydroxyproline
  MOD_RES (254)..(254): Hydroxyproline
  MOD_RES (257)..(257): Hydroxyproline
  MOD_RES (260)..(260): Hydroxyproline
  MOD_RES (263)..(263): Hydroxyproline
  MOD_RES (266)..(266): Hydroxyproline
  MOD_RES (269)..(269): Hydroxyproline
  MOD_RES (272)..(272): Hydroxyproline
  MOD_RES (275)..(275): Hydroxyproline
  MOD_RES (278)..(278): Hydroxyproline
  MOD_RES (281)..(281): Hydroxyproline
  MOD_RES (284)..(284): Hydroxyproline
  MOD_RES (287)..(287): Hydroxyproline
  MOD_RES (290)..(290): Hydroxyproline
  MOD_RES (293)..(293): Hydroxyproline
  MOD_RES (296)..(296): Hydroxyproline
  MOD_RES (299)..(299): Hydroxyproline
PPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPGPPG

SEQ ID NO: 9 - protein, 20 aa, Artificial Sequence
  Description of Artificial Sequence Synthetic His tag
  MISC_FEATURE (1)..(20): This sequence may encompass 2-20 residues
HHHHHHHHHHHHHHHHHHHH

SEQ ID NO: 10 - protein, 15 aa, Artificial Sequence
  Description of Artificial Sequence Synthetic His tag
  MISC_FEATURE (1)..(15): This sequence may encompass 5-15 residues
HHHHHHHHHHHHHHH

SEQ ID NO: 11 - protein, 18 aa, Artificial Sequence
  Description of Artificial Sequence Synthetic His tag
  MISC_FEATURE (1)..(18): This sequence may encompass 5-18 residues
HHHHHHHHHHHHHHHHHH

SEQ ID NO: 12 - protein, 16 aa, Artificial Sequence
  Description of Artificial Sequence Synthetic His tag
  MISC_FEATURE (1)..(16): This sequence may encompass 5-16 residues
HHHHHHHHHHHHHHHH

SEQ ID NO: 13 - protein, 14 aa, Artificial Sequence
  Description of Artificial Sequence Synthetic His tag
  MISC_FEATURE (1)..(14): This sequence may encompass 5-14 residues
HHHHHHHHHHHHHH

SEQ ID NO: 14 - protein, 13 aa, Artificial Sequence
  Description of Artificial Sequence Synthetic His tag
  MISC_FEATURE (1)..(13): This sequence may encompass 5-13 residues
HHHHHHHHHHHHH

SEQ ID NO: 15 - protein, 12 aa, Artificial Sequence
  Description of Artificial Sequence Synthetic His tag
  MISC_FEATURE (1)..(12): This sequence may encompass 5-12 residues
HHHHHHHHHHHH

SEQ ID NO: 16 - protein, 11 aa, Artificial Sequence
  Description of Artificial Sequence Synthetic His tag
  MISC_FEATURE (1)..(11): This sequence may encompass 5-11 residues
HHHHHHHHHHH

SEQ ID NO: 17 - protein, 10 aa, Artificial Sequence
  Description of Artificial Sequence Synthetic His tag
  MISC_FEATURE (1)..(10): This sequence may encompass 5-10 residues
HHHHHHHHHH

SEQ ID NO: 18 - protein, 12 aa, Artificial Sequence
  Description of Artificial Sequence Synthetic His tag
  MISC_FEATURE (1)..(12): This sequence may encompass 6-12 residues
HHHHHHHHHHHH

SEQ ID NO: 19 - protein, 11 aa, Artificial Sequence
  Description of Artificial Sequence Synthetic His tag
  MISC_FEATURE (1)..(11): This sequence may encompass 6-11 residues
HHHHHHHHHHH

SEQ ID NO: 20 - protein, 10 aa, Artificial Sequence
  Description of Artificial Sequence Synthetic His tag
  MISC_FEATURE (1)..(10): This sequence may encompass 7-10 residues
HHHHHHHHHH

SEQ ID NO: 21 - protein, 9 aa, Artificial Sequence
  Description of Artificial Sequence Synthetic 9xHis tag
HHHHHHHHH

(19) United States
(12) Patent Application Publication
(10) Pub. No.: US 2020/0184381 A1
(43) Pub. Date: Jun. 11, 2020

(54) METHODS AND SYSTEMS FOR ENGINEERING COLLAGEN

(71) Applicant: Geltor, Inc., San Leandro, CA (US)

(72) Inventors: Anton V. Persikov, Princeton, NJ (US); Nikolay Ouzounov, Alameda, CA (US); Alexander Lorestani, Oakland, CA (US)

(21) Appl. No.: 16/462,196

(22) PCT Filed: Nov. 19, 2018
(86) PCT No.: PCT/US2018/061882

§ 371 Date: May 17, 2019

Related U.S. Application Data
(60) Provisional application No. 62/590,183, filed on Nov. 22, 2017.

(51) Int. Cl. G06N 20/20; G06N 20/10; C07K 14/78; G16B 30/00; G16B 40/20; G06N 5/00

(52) U.S. Cl. CPC G06N 20/20; G06N 20/10; G06N 5/003; G16B 30/00; G16B 40/20; C07K 14/78

(57) ABSTRACT

This disclosure describes methods and systems for engineering and manufacturing collagen-based biomaterials. The methods and systems combine synthetic biology, fermentation, material science and machine learning. Collagen molecules or collagen based materials obtained from using the methods have desired physical or chemical properties such as melting temperature, stiffness, or elasticity. The obtained collagen molecules and sequences are also disclosed.

### CROSS REFERENCE TO RELATED APPLICATIONS

This application claims benefit and priority to U.S. Provisional Patent Application No. 62/590,183, entitled: METHODS AND SYSTEMS FOR ENGINEERING COLLAGEN, filed Nov. 22, 2017, which is herein incorporated by reference in its entirety for all purposes.

### BACKGROUND

The present disclosure relates to collagen and collagen derived materials. Methods and systems for engineering collagen using machine learning models and genetic engineering techniques are also disclosed.

Collagen is the most abundant protein in animals and is deployed as a biomaterial in technical and consumer markets. The physical-chemical and structural properties of collagen are desirable for biomaterials and include mechanical strength, resistance to proteases, and the ability to associate into fibrils. Collagen's denatured form, gelatin, is known to form strong, transparent gels and flexible films, making it a desirable material in a wide range of commercial applications.

Currently, most collagen biomaterials are obtained from animal sources, such as pig, cow or fish. However, there is a growing demand for animal-free collagen products driven by the inconsistency of animal-derived materials, the inability to tune their properties, and changing consumer preferences. Further, the rapidly increasing demand for collagen-based products in certain markets has unmasked the need for a sustainable and scalable collagen biomaterial manufacturing platform.

This disclosure provides industrial processes and systems for engineering collagen and collagen derived materials using machine learning and genetic engineering techniques. The collagen can be designed to possess desired physical or chemical properties of gelatin product, providing applications in a wide range of industries such as health care, cosmetics, food. The collagen can be manufactured using genetic engineering techniques and microorganism expression systems without using animal products.

### SUMMARY

One aspect of the disclosure provides methods for engineering one or more collagen molecules. The method includes (a) obtaining, using a machine learning model and by a computer system comprising one or more processors and system memory, a set of target data comprising frequencies of amino acid residues in one or more target collagen sequences, wherein the set of target data is predicted by the machine learning model to be associated with at least one physical or chemical property meeting a criterion, wherein the machine learning model was obtained by: (i) receiving a set of training data comprising frequencies of amino acid residues in a plurality of training collagen sequences and physical or chemical property data of the at least one physical or chemical property associated with the plurality of training collagen sequences; and (ii) training the machine learning model by fitting the machine learning model to the set of training data, wherein the trained machine learning model is configured to receive as input amino acid data of a test collagen sequence and predict at least one value of the at least one physical or chemical property associated with the test collagen sequence. The method also includes: (b) determining, by the computer system, one or more collagen sequences corresponding to the set of target data; (c) producing one or more polynucleotides encoding the one or more collagen sequences; and (d) expressing, on a protein production platform, the one or more polynucleotides to produce one or more collagen molecules comprising the one or more collagen sequences.

In some implementations, the frequencies of amino acid residues indicates intra-sequence variation of amino acid trimers in the plurality of collagen sequences. In some implementations, the frequencies of amino acid residues include: (a) a frequency for each of a plurality of different amino acids as residues at X positions of X-Y-Gly trimers in each training collagen sequence, and (b) a frequency for each of the different plurality of amino acids as residues at Y positions of the X-Y-Gly trimers in the training collagen sequence. In some implementations, the plurality of different amino acids includes 20 standard amino acids naturally occurring in organisms.

In some implementations, the plurality of amino acids further includes post-translational modifications of the 20 standard amino acids. In some implementations, the plurality of amino acids consists of a subset of 20 standard amino acids and post-translationally modified amino acids of the subset.

In some implementations, the set of training data is generated using a main collagen domain with an uninterrupted (X-Y-Gly)ₙ repeating sequence.

In some implementations, the set of training data includes lengths of the plurality of training collagen sequences or fragments thereof.

In some implementations, the frequencies of amino acid residues include: frequencies of amino acid residues in two or more regions of each training collagen sequence. In some implementations, the frequencies of amino acid residues include: (a) a frequency for each of a plurality of different amino acids at X positions of X-Y-Gly trimers in a first region of each training collagen sequence, (b) a frequency for each of a plurality of different amino acids at Y positions of X-Y-Gly trimers in the first region of each training collagen sequence, (c) a frequency for each of the plurality of different amino acids at the X positions of the X-Y-Gly trimers in a second region of each training collagen sequence, and (d) a frequency for each of the plurality of different amino acids at the Y positions of the X-Y-Gly trimers in the second region of each training collagen sequence.

In some implementations, the machine learning model includes a support vector machine. In some implementations, the support vector machine has a linear kernel. In some implementations, the support vector machine has a nonlinear kernel. In some implementations, training the machine learning model includes applying a linear support vector machine and a weight vector analysis to reduce dimensionality of a feature space.

In some implementations, training the machine learning model includes applying a principal component analysis to reduce dimensionality of feature space.

In some implementations, the machine learning model includes a random forest model. In some implementations, the machine learning model includes a neural network model. In some implementations, the machine learning model includes a general linear model.

In some implementations, the plurality of training collagen sequences includes a plurality of collagen sequences.

In some implementations, the plurality of training collagen sequences includes a plurality of gelatin sequences.

In some implementations, the at least one physical or chemical property is selected from a group consisting of: melting or gelling temperature, stiffness, elasticity, oxygen release rate, clarity, turbidity, ultraviolet blockage or absorption, viscosity, solubility, water content or hydration, resistance to protease, and ability to associate into fibrils. In some implementations, the at least one physical or chemical property includes two or more physical or chemical properties.

In some implementations, the one or more polynucleotides include recombinant polynucleotides. In some implementations, the one or more polynucleotides include synthesized polynucleotides.

In some implementations, the one or more collagen molecules produced in (d) include recombinant collagen molecules.

In some implementations, the method further includes manufacturing, using the one or more collagen molecules produced in (e), gelatin materials or collagen derivatives.

Another aspect of the disclosure provides a non-naturally occurring collagen polypeptide comprising: (a) an amino acid sequence of a secretion tag selected from the group consisting of DsbA, pelB, OmpA, TolB, MalE, lpp, TorA, and HylA; and (b) a plurality of X-Y-Gly trimers, wherein (i) amino acids at X positions of the X-Y-Gly trimers are selected from a group consisting of: alanine, cysteine, aspartic acid, glutamic acid, phenylalanine, glycine, histidine, isoleucine, lysine, leucine, methionine, asparagine, proline, pyrrolysine, glutamine, arginine, serine, threonine, selenocysteine, valine, tryptophan, tyrosine, and post-translational modifications therefrom, (ii) amino acids at Y positions of the X-Y-Gly trimers are selected from a group consisting of: alanine, cysteine, aspartic acid, glutamic acid, phenylalanine, glycine, histidine, isoleucine, lysine, leucine, methionine, asparagine, proline, pyrrolysine, glutamine, arginine, serine, threonine, selenocysteine, valine, tryptophan, tyrosine, and post-translational modifications therefrom, and (iii) the non-naturally occurring collagen polypeptide was predicted by a machine learning model to be associated with at least one physical or chemical property meeting a criterion.

In some implementations, the non-naturally occurring collagen polypeptide further includes amino acid sequences selected from the group consisting of a histidine tag, green fluorescent protein, protease cleavage site, and a beta-lactamase protein.

In some implementations, the machine learning model was obtained by: (i) receiving a set of training data including frequencies of amino acid residues in a plurality of training collagen sequences and physical or chemical property data of at least one physical or chemical property associated with the plurality of training collagen sequences; and (ii) training the machine learning model by fitting the machine learning model to the set of training data, wherein the trained machine learning model is configured to receive as input amino acid data of a test collagen sequence and predict at least one value of the at least one physical or chemical property associated with the test collagen sequence. In some implementations, the frequencies of amino acid residues include: (a) a frequency for each of a plurality of different amino acids as residues at the X positions of X-Y-Gly trimers in each training collagen or gelatin repeating sequence, and (b) a frequency for each of the plurality of different amino acids as residues at the Y positions of the X-Y-Gly trimers in the training collagen or gelatin repeating sequence.

In some implementations, one or more of the amino acids at the X or Y positions of the X-Y-Gly trimers include (2S,4R)-4-hydroxyproline.

In some implementations, the amino acids at the X or Y positions of the X-Y-Gly trimers are selected from a group consisting of: alanine, cysteine, aspartic acid, glutamic acid, phenylalanine, glycine, histidine, isoleucine, lysine, leucine, methionine, asparagine, proline, glutamine, arginine, serine, threonine, valine, tryptophan, tyrosine, and post-translational modifications therefrom.

In some implementations, the non-naturally occurring collagen polypeptide is capable of forming a homomeric or heteromeric triple helix.

In some implementations, the at least one physical or chemical property includes melting or gelling temperature. In some implementations, the at least one physical or chemical property includes stiffness.

In some implementations, the at least one physical or chemical property includes elasticity.

In some implementations, the at least one physical or chemical property includes oxygen release rate.

In some implementations, the at least one physical or chemical property includes clarity.

In some implementations, the at least one physical or chemical property includes ultraviolet blockage or absorption.

In some implementations, the non-naturally occurring collagen polypeptide was produced by: (a) obtaining, using the machine learning model, a set of target data including frequencies of amino acid residues in one or more target collagen sequences, wherein the set of target data is predicted by the machine learning model to be associated with at least one physical or chemical property meeting a criterion; (b) determining one or more collagen sequences corresponding to the set of target data; and (c) producing the non-naturally occurring collagen polypeptide including the one or more collagen sequences.

An additional aspect of the disclosure provides a non-naturally occurring gelatin polypeptide including: (a) an amino acid sequence of a secretion tag selected from the group consisting of DsbA, pelB, OmpA, TolB, MalE, lpp, TorA, and HylA; and (b) a plurality of X-Y-Gly trimers, where (i) amino acids at X positions of the X-Y-Gly trimers are selected from a group consisting of: alanine, cysteine, aspartic acid, glutamic acid, phenylalanine, glycine, histidine, isoleucine, lysine, leucine, methionine, asparagine, proline, pyrrolysine, glutamine, arginine, serine, threonine, selenocysteine, valine, tryptophan, tyrosine, and post-translational modifications therefrom, (ii) amino acids at Y positions of the X-Y-Gly trimers are selected from a group consisting of: alanine, cysteine, aspartic acid, glutamic acid, phenylalanine, glycine, histidine, isoleucine, lysine, leucine, methionine, asparagine, proline, pyrrolysine, glutamine, arginine, serine, threonine, selenocysteine, valine, tryptophan, tyrosine, and post-translational modifications therefrom, and (iii) the non-naturally occurring gelatin polypeptide was predicted by a machine learning model to be associated with at least one physical or chemical property meeting a criterion.

Computer systems and computer program products for practicing the methods and making the compounds are also disclosed.

One aspect of the disclosure provides computer program product including a non-transitory machine readable medium storing program code that, when executed by one or more processors of a computer system, causes the computer system to implement a method for engineering one or more collagen molecules, said program code including: code for receiving a set of training data including frequencies of amino acid residues in a plurality of training collagen sequences and physical or chemical property data of at least one physical or chemical property associated with the plurality of training collagen sequences; and code for training a machine learning model by fitting the machine learning model to the set of training data, wherein the trained machine learning model is configured to receive as input amino acid data of a test collagen sequence and predict at least one value of the at least one physical or chemical property associated with the test collagen sequence.

In some implementations, the program code further includes: code for determining, using the machine learning model, a set of target data including frequencies of amino acid residues in one or more target collagen sequences, wherein the set of target data is predicted by the machine learning model to be associated with the at least one physical or chemical property meeting a criterion; and code for determining one or more collagen sequences corresponding to the set of target data.

Another aspect of the disclosure provides a computer system, including: one or more processors; system memory; and one or more computer-readable storage media having stored thereon computer-executable instructions that, when executed by the one or more processors, cause the computer system to implement a method for engineering one or more collagen molecules. The one or more processors are configured to: receive a set of training data including frequencies of amino acid residues in a plurality of training collagen sequences and physical or chemical property data of at least one physical or chemical property associated with the plurality of training collagen sequences; and train a machine learning model by fitting the machine learning model to the set of training data, wherein the trained machine learning model is configured to receive as input amino acid data of a test collagen sequence and predict at least one value of the at least one physical or chemical property associated with the test collagen sequence.

In some implementations, the one or more processors are further configured to: determine, using the machine learning model, a set of target data including frequencies of amino acid residues in one or more target collagen sequences, wherein the set of target data is predicted by the machine learning model to be associated with the at least one physical or chemical property meeting a criterion; and determine one or more collagen sequences corresponding to the set of target data.

These and other features of the present disclosure will become more fully apparent from the following description and appended claims, or may be learned by the practice of the disclosure as set forth hereinafter.

### BRIEF DESCRIPTION OF THE DRAWINGS

FIG. 1 illustrates a workflow for engineering collagen molecules according to some implementations.

FIG. 2 illustrates how a feature vector is generated and labeled by the physical properties of collagen according to some implementations.

FIG. 3 graphically illustrates how a support vector machine (SVM) can be used to model collagen sequences and properties.

FIG. 4 shows a simplified regression tree that can be used to model collagen sequences and properties.

FIG. 5 illustrates an ensemble of regression trees to form a random forest in the training phase of a random forest model.

FIG. 6 illustrates applying the random forest model to determine the property of a collagen in a test phase.

FIG. 7 shows an exemplary digital device that can be implemented according to some embodiments.

FIG. 8 depicts the physiological state difference between switched and unswitched cells. A) Unswitched Escherichia coli cells. B) Same Escherichia coli population as figure A but has undergone the physiological switch. C) Phase contrast of switched Escherichia coli cell containing cytoplasmic RFP and periplasmic GFP. D) Fluorescent imaging of cell in figure C illustrates targeted protein localization.

FIG. 9 depicts enhanced protein production in switched cells. A-B) Target protein for T7 inducible protein production is periplasmic expressed GFP, produced in Escherichia coli BL21. The same population of cells was used and induced at OD 1.1. A) Protein ladder (lane 1), IPTG induced protein production (lane 2), IPTG induced protein production with physiological switch (lane 3). B) Two vials of the cell GFP induced cultures with IPTG only on left and IPTG+Switch on right. C) Expression of a 22 KD collagen using switched cells showing protein ladder (lane 1), supernatant after protein production (lane 2), cell pellet (lane 3).

FIG. 10 depicts a time lapse of Escherichia coli cell switching over time.

FIG. 11 illustrates other organisms undergoing the physiological switch. A) Agrobacterium tumefaciens normal physiology. B) Agrobacterium tumefaciens switched physiology. C) Pseudomonas aeruginosa PAO1 normal physiology. D) Pseudomonas aeruginosa PAO1 switched physiology. E) Brevundimonas diminuta normal physiology. F) Brevundimonas diminuta switched physiology. G) Agrobacterium tumefaciens normal physiology. H) Agrobacterium tumefaciens switched physiology.

### DETAILED DESCRIPTION

This disclosure describes methods and systems for engineering and manufacturing collagen-based biomaterials. The methods combine molecular biology, fermentation, material science and machine learning. Collagen-based materials obtained from using the methods have desired physical or chemical properties such as melting temperature, stiffness or elasticity. The obtained collagen molecules and sequences are also disclosed.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not intended to limit the disclosure.

Unless defined otherwise herein, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice or testing of the embodiments disclosed herein, some methods and materials are described.

The terms defined immediately below are more fully described by reference to the specification as a whole. It is to be understood that this disclosure is not limited to the particular methodology, protocols, and reagents described, as these may vary, depending upon the context they are used by those of skill in the art.

As used in this specification and appended claims, the singular forms “a”, “an”, and “the” include plural referents unless the content and context clearly dictates otherwise. Thus, for example, reference to “a device” includes a combination of two or more such devices, and the like. Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected).

### I. Definitions

As used herein the term “about” refers to ±10%.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Collagen is a structural protein in the extracellular space in the various connective tissues in animal bodies. Collagen consists of three polypeptide chains wound together to form triple-helices.

The quaternary structure of natural collagen is a triple helix typically composed of three polypeptides. The term “procollagen” as used herein refers to polypeptides produced by cells that can be processed to naturally occurring collagen.

Gelatin is an irreversibly denatured form of collagen, wherein the hydrolysis results in the reduction of protein fibrils into smaller peptides, which have broad molecular weight ranges associated with physical and chemical methods of denaturation, based on the process of hydrolysis. Collagen can be treated with acid, base or heat to prepare gelatin. While not wishing to be bound by theory or mechanism, treatment of collagen with acid, base or heat is thought to denature the collagen polypeptides. Aqueous denatured collagen solutions form reversible gels used in foods, cosmetics, pharmaceuticals, industrial products, medical products, laboratory culture growth media, and many other applications.

The term “collagen sequence” is used herein to refer to an amino acid sequence of a collagen polypeptide, which can bind with two other polypeptides to form a triple-helix of a collagen molecule. The term is also used to refer to an amino acid sequence found in gelatin protein. In this latter use, the term is interchangeable with “gelatin sequence.”

Random Forests Model—Random Forests is a method for multiple regression or classification using an ensemble of decision trees. Each decision tree of the ensemble is trained with a subset of data from the available training data set. At each node of a decision tree, a number of variables are randomly selected from all of the available variables to train the decision rule. When applying a train Random Forest, test data are provided to the decision trees of the Random Forest ensemble, and the final outcome is based on a combination of the outcomes of the individual decision trees. For classification decision trees, the final class may be a majority or a mode of the outcomes of all the decision trees. For regression decision trees (or simply regression trees), the final value can be a mean, a mode, or a median. Examples and details of Random Forest methods are further described hereinafter.

Support vector machines (SVMs) are machine learning tools with associated learning algorithms for classification and regression analysis. A classification SVM, like other machine learning classifiers, takes a set of input data and predicts, for each given input, which of two possible classes forms the output. Given a set of training examples, each marked as belonging to one of two categories, a classification SVM training algorithm builds a model that assigns new examples into one category or the other. An SVM is a representation of the examples as points in multi-dimensional feature space, mapped so that the examples of the separate categories are divided by a clear gap that is as wide as possible, which is implemented by maximizing the distance between data points and a hyperplane separating the two categories. In addition to performing linear classification, SVMs can efficiently perform a non-linear classification using a kernel trick to implicitly map inputs into higher-dimensional feature spaces.

A regression SVM takes as input one or more independent variables (IVs) of an individual and predicts values of a dependent variable (DV) of the individual based on the relation between the IVs and the DV in training data. Given a set of training individual a regression SVM training algorithm builds a model that finds a function relating IVs and the DV. The model limits prediction errors in a defined range, penalizing prediction errors only when the errors exceed the range.

The terms “protein,” “polypeptide” and “peptide” are used interchangeably to denote a polymer of at least two amino acids covalently linked by an amide bond, regardless of length or post-translational modification (e.g., glycosylation, phosphorylation, lipidation, myristilation, ubiquitination, etc.). In some cases, the polymer has at least about 30 amino acid residues, and usually at least about 50 amino acid residues. More typically, they contain at least about 100 amino acid residues. It is not intended that the present invention be limited to amino acid sequences of any specific length. The terms include compositions conventionally considered to be fragments of full-length proteins or peptides. Included within this definition are D- and L-amino acids, and mixtures of D- and L-amino acids. The polypeptides described herein are not restricted to the genetically encoded amino acids. Indeed, in addition to the genetically encoded amino acids, the polypeptides described herein may be made up of, either in whole or in part, naturally-occurring and/or synthetic non-encoded amino acids. In some embodiments, a polypeptide is a portion of the full-length ancestral or parental polypeptide, containing amino acid additions or deletions (e.g., gaps), and/or substitutions as compared to the amino acid sequence of the full-length parental polypeptide, while still retaining functional activity (e.g., catalytic activity).

As used herein, the term “wild-type” or “wildtype” (WT) refers to naturally-occurring proteins (e.g., non-recombinant proteins). A substrate or ligand that reacts with a wild-type biomolecule is sometimes considered a “native” substrate or ligand.

The term “sequence” is used herein to refer to the order and identity of any biological sequences including but not limited to a whole genome, whole chromosome, chromosome segment, collection of gene sequences for interacting genes, gene, nucleic acid sequence, protein, peptide, polypeptide, polysaccharide, etc. In some contexts, a “sequence” refers to the order and identity of amino acid residues in a protein (i.e., a protein sequence or protein character string) or to the order and identity of nucleotides in a nucleic acid (i.e., a nucleic acid sequence or nucleic acid character string). A sequence may be represented by a character string. A “nucleic acid sequence” refers to the order and identity of the nucleotides comprising a nucleic acid. A “protein sequence” refers to the order and identity of the amino acids comprising a protein or peptide.

Two nucleic acids are “recombined” when sequences from each of the two nucleic acids are combined to produce progeny nucleic acid(s). Two sequences are “directly” recombined when both of the nucleic acids are substrates for recombination.

A “dependent variable” (“DV”) represents an output or effect, or is tested to see if it is the effect. The “independent variables” (“IVs”) represent the inputs or causes, or are tested to see if they are the cause. A dependent variable may be studied to see if and how much it varies as the independent variables vary.

In the simple stochastic linear model

yᵢ=a+bxᵢ+eᵢ

where the term yᵢ is the i^{th} value of the dependent variable and xᵢ is i^{th} value of the independent variable (IV). The term eᵢ is known as the “error” and contains the variability of the dependent variable not explained by the independent variable.

An independent variable (IV) is also known as a “predictor variable”, “regressor”, “controlled variable”, “manipulated variable”, “explanatory variable”, or “input variable”.

The term “coefficient” refers to a scalar value multiplied by a dependent variable or an expression containing a dependent variable.

The phrase “training set” refers to a set of collagen sequence and property data or observations that one or more models are fitted to and built upon. For instance, for a protein machine learning model, a training set comprises amino acid frequencies for an initial collagen protein library and one or more physical or chemical properties.

The term “observation” is information about protein or other biological entity that may be used in a training set for generating a model such as a machine learning model. The term “observation” may refer to any sequenced and assayed biological molecules, including protein variants. Generally, the more observations employed to create a machine learning model, the better the predictive power of that machine learning model.

The phrase “cross validation” refers to a method for testing the generalizability of a model's ability to predict the value of the dependent variable. The entire data set with known labels is randomly split into training and validation sets. The method prepares a model using the training set, and tests the model error using the validation set. This process is repeated multiple times to reduce any possible split bias.

The terms “regression” and “regression analysis” refer to techniques used to understand which of the independent variables are related to the dependent variable, and to explore the forms of these relationships. In restricted circumstances, regression analysis can be used to infer causal relationships between the independent and dependent variables. It is a statistical technique for estimating the relationships among variables. It includes many techniques for modeling and analyzing several variables, when the focus is on the relationship between a dependent variable and one or more independent variables. More specifically, regression analysis helps one understand how the typical value of the dependent variable changes when any one of the independent variables is varied, while the other independent variables are held fixed. Regression techniques may be used to generate machine learning models from training sets comprising multiple observations, which may contain amino acid frequencies and physical or chemical property information.

“Partial Least Squares” (“PLS”) is a family of methods that finds a linear regression model by projecting predicted variables (e.g., activities) and the observable variables (e.g., sequences) to a new space. PLS is also known as “projection to latent structures.” Both the X (independent variables) and Y (dependent variables) data are projected to new spaces. PLS is used to find the fundamental relations between two matrices (X and Y). A latent variable model is used to model the covariance structures in the X and Y spaces. A PLS model will try to find the multi-dimensional direction in the X space that explains the maximum multi-dimensional variance direction in the Y space. PLS regression is particularly useful when the matrix of predictors has more variables than observations, and when there is multi-collinearity among X values.

In a regression model, the dependent variable is related to independent variables by a sum of terms. Each term includes a product of an independent variable and an associated regression coefficient. In the case of a purely linear regression model, the regression coefficients are given by β in the following form of expression:

yᵢ=β₁xᵢ₁+ . . . +βₚxᵢₚ+εᵢ=xᵢ^{T}β+εᵢ

where yᵢ is the dependent variable, the xᵢ are the independent variables, εᵢ is the error variable, and T denotes the transpose, that is the inner product of the vectors xᵢ and β.

The phrase “principal component analysis” (“PCA”) refers to a mathematical procedure that uses an orthogonal transformation to convert a set of observations of possibly correlated variables into a set of values of linearly uncorrelated variables called “principal components.” The number of principal components is less than or equal to the number of original variables. This transformation is defined in such a way that the first principal component has the largest possible variance (that is, accounts for as much of the variability in the data as possible), and each succeeding component in turn has the highest variance possible under the constraint that it be orthogonal to (i.e., uncorrelated with) the preceding components.

A “neural network” is a model containing an interconnected group of processing elements or “neurons” that process information using a connectionist approach to computation. Neural networks are used to model complex relationships between inputs and outputs and/or to find patterns in data. Most neural networks process data in a non-linear, distributed, parallel fashion. In most cases, neural networks are adaptive systems that change their structure during a learning phase. Functions are performed collectively and in parallel by the processing elements, rather than using a clear delineation of subtasks to which various units are assigned.

Generally, a neural network involves a network of simple processing elements that exhibit complex global behavior determined by the connections between the processing elements and element parameters. Neural networks are used with algorithms designed to alter the strength of the connections in the network to produce a desired signal flow. The strength is altered during training or learning.

The term “expression vector” or “vector” as used herein refers to a nucleic acid assembly that is capable of directing an expression of an exogenous gene. The expression vector may include a promoter which is operably linked to the exogenous gene, restriction endonuclease sites, nucleic acids that encode one or more selection markers, and other nucleic acids useful in the practice of recombinant technologies.

The term “fibroblast” as used herein refers to a cell that synthesizes procollagen and other structural proteins. Fibroblasts are widely distributed in the body and found in skin, connective tissue and other tissues.

The term “fluorescent protein” is a protein that is commonly used in genetic engineering technologies used as a reporter of expression of an exogenous polynucleotide. The protein when exposed to ultraviolet or blue light fluoresces and emits a bright visible light. Proteins that emit green light is green fluorescent protein (GFP) and proteins that emit red light is red fluorescent protein (RFP)

The term “gene” as used herein refers to a polynucleotide that encodes a specific protein, and which may refer to the coding region alone or may include regulatory sequences preceding (5′ non-coding sequences) and following (3′ non-coding sequences) the coding sequence.

The term “histidine tag” is a 2-30 contiguous series of histidine residues on a recombinant polypeptide.

The term “host cell” is a cell that is engineered to express an introduced exogenous polynucleotide.

The term “lactamase” as used herein refer to enzymes that hydrolyze antibiotics that contain a lactam (cyclic amide) moiety. “Beta-lactamase” or “β-lactamase” is a class of enzymes that hydrolyzes antibiotics that contain a β-lactam moiety.

The term “non-naturally occurring” as used herein refers to collagen or gelatin that is not normally found in nature. The non-naturally occurring collagen is in one embodiment a truncated collagen. Other non-naturally occurring collagen polypeptides include chimeric collagens. A chimeric collagen is a polypeptide wherein one portion of a collagen polypeptide is contiguous with a portion of a second collagen polypeptide. For example, a collagen molecule comprising a portion of a jellyfish collagen contiguous with a portion of a Tilapia collagen is a chimeric collagen. In another embodiment, the non-naturally occurring collagen comprises a fusion polypeptide that includes additional amino acids such as a secretion tag, histidine tag, green fluorescent protein, protease cleavage site, GEK repeats, GDK repeats, and/or beta-lactamase.

The term “protease cleavage site” is an amino acid sequence that is cleaved by a specific protease.

The term “secretion tag” or “signal peptide” refers to an amino acid sequence that recruits the host cell's cellular machinery to transport an expressed protein to a particular location or cellular organelle of the host cell.

The term “truncated collagen” refers to a monomeric polypeptide that is smaller than a full-length collagen wherein one or more portions of the full-length collagen are not present. Collagen polypeptides are truncated at the C-terminal end, the N-terminal end, or truncated by removal of internal portion(s) of the full-length collagen polypeptide.

### II. Introduction

Native collagen is a triple-helix comprising three left-handed polyproline II-like helical chains, wound around each other to form a tightly packed right-handed superhelix. Only Gly residues can be accommodated without distortion as every third residue near the center of this supercoiled helix. This generates a repeating sequence of the form (X-Y-Gly)ₙ. The X and Y positions can accommodate any amino acid, but about 20% of these positions in natural fibrillary collagens are occupied by imino acids. Proline (Pro) residues are incorporated into both the X and Y positions during biosynthesis, and this is followed by enzymatic post-translational hydroxylation of prolines in the Y positions to form hydroxyproline (Hyp). (Pro-Hyp-Gly)ₙ is the most stabilizing tripeptide unit (or trimmer repeat) present in collagen, and also represents the most common sequence. Persikov A V, Ramshaw J A, Kirkpatrick A, Brodsky B. (2000) Amino acid propensities for the collagen triple-helix. Biochemistry. 39(48): 14960-7.

Natural collagens are synthesized in a procollagen form, with globular propeptides on each end of a central triple-helix. Self-association and disulfide cross-linking of three C-propeptides are responsible for the initial events of chain selection and trimer formation, whereas subsequent events include nucleation and zipper-like folding of the triple-helix domain. After cleavage of the propeptides, the rod-like triple-helical molecules in the matrix self-associate in a staggered array, forming fibrils and interacting with other matrix molecules to provide the strength, flexibility, or compression required for each tissue. Persikov A V, Ramshaw J A, Kirkpatrick A, Brodsky B. (2002) Peptide investigations of pairwise interactions in the collagen triple-helix. J Mol Biol. 316(2): 385-94.

Once folded, collagen is not cross-linked anymore. Therefore, thermal unfolding of collagen is irreversible, and the randomly coiled collagen molecule does not fold back into a native triple-helix with properly aligned chains at any cooling procedure. Unfolded collagen chains will, however, partially recover in triple-helical fragments, while chain misalignment will result in dangled single-chain ends of various lengths. These ends, in turn, will associate into short triple-helical fragments, making longer aggregates, compiling network-like macroscopic structures. These re-folded collagen structures may exist in two states: a dilute solution and a coacervate consisting of a concentrated form. When the concentration is sufficiently high and the temperature is low enough, the solution loses its fluidity to become a gelatin. The phase separation temperature (gelatin melting temperature) depends on the original collagen sequence, as well as cooling procedure and gelatin water content. Modulation of collagen sequences can produce gelatins with a wide range of physical-chemical properties, including variable stiffness and melting temperature (Tm).

Currently, most collagen biomaterials are obtained from animal sources, such as pig, cow or fish. However, there is a growing demand for animal-free collagen products driven by the inconsistency of animal-derived materials, the inability to tune their properties, and changing consumer preferences. Further, the rapidly increasing demand for collagen-based products in certain markets has unmasked the need for a sustainable and scalable collagen biomaterial manufacturing platform.

Since the structural and physical properties of gelatin are dependent on the stability of the collagen triple-helix, it is useful to use basic principles of triple-helix stability to understand its effect on the physical-chemical properties of gelatin.

Previous studies of model collagen mimetic peptides led to understanding of which combinations of charged and hydrophobic residues control the thermal stability of collagen molecule fragments and their ability to form higher-ordered structures. However, the combination of amino acids determining thermal stability and mechanical properties of collagen-based biomaterials remains unknown. This disclosure describes approaches to collagen-based biomaterial design and manufacturing which combines synthetic biology, machine learning, material science and fermentation.

### III Workflow for Engineering Collagen or Gelatin Proteins

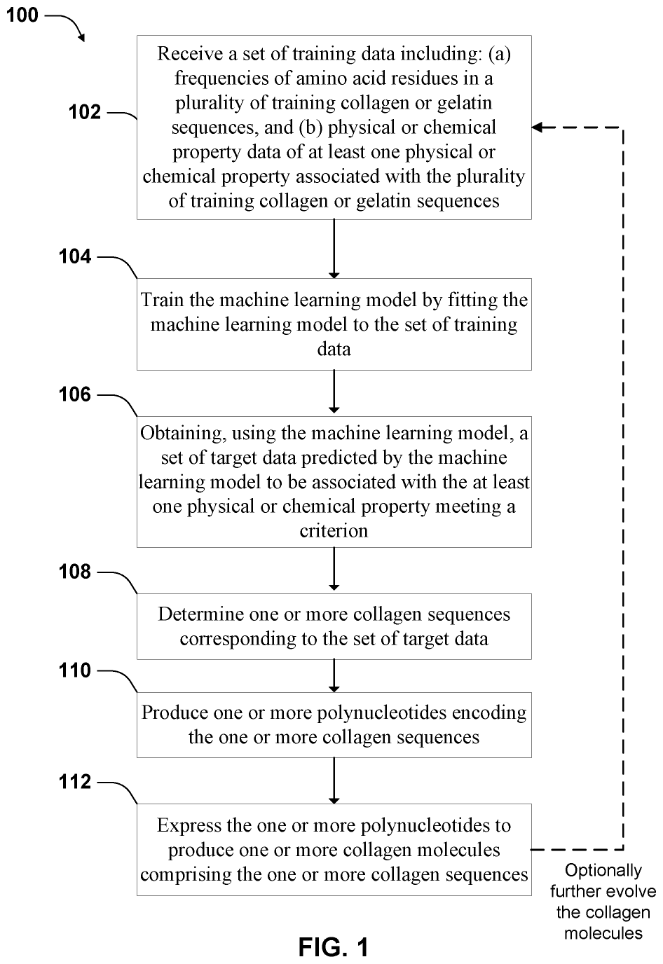

One aspect of the disclosure provides methods for engineering collagen or gelatin molecules. The methods use machine learning models to design collagen protein sequences to form gelatin product with desired properties. FIG. 1 illustrates a workflow, process 100, according to some implementations. Process 100 involves receiving a set of training data that includes information about the amino acid content in each of a plurality of training collagen sequences. See block 102. In some implementations, the information provides frequencies of the various amino acids found in the X and Y-positions of collagen sequences. In addition to information about amino acid content, the training data set includes physical or chemical property data of at least one physical or chemical property associated with the plurality of training collagen sequences. For example, each training set member includes a value of elasticity, such as a value of Young's modulus, and amino acid frequencies for a single gelatin molecule. Process 100 also involves training a machine learning model by fitting the machine learning model to the set of training data. See block 104.

To create a training set, some implementations involve producing a set of recombinant collagens with variable sequences. In some implementations, the training set includes naturally occurring collagen sequences and/or synthetic sequences incorporating various charged residues (Lys, Arg, Glu, Asp), hydrophobic residues (Leu, Ile, Phe), and other naturally occurring amino acids. In some implementations, the naturally occurring nucleic amino acids include the 20 standard amino acids (alanine, cysteine, aspartic acid, glutamic acid, phenylalanine, glycine, histidine, isoleucine, lysine, leucine, methionine, asparagine, proline, glutamine, arginine, serine, threonine, valine, tryptophan, tyrosine). In some implementations, the naturally occurring amino acids also include the two nonstandard amino acids (pyrrolysine and selenocysteine). In some implementations, the amino acids include post-translationally modified amino acids, e.g., hydroxyproline derived from proline and hydroxylysine derived from lysine. In some implementations, one or more of the amino acids include (2S,4R)-4-hydroxyproline. In some implementations, one or more of the amino acids include synthetic forms of hydroxyprolines other than (2S,4R)-4-hydroxyproline.

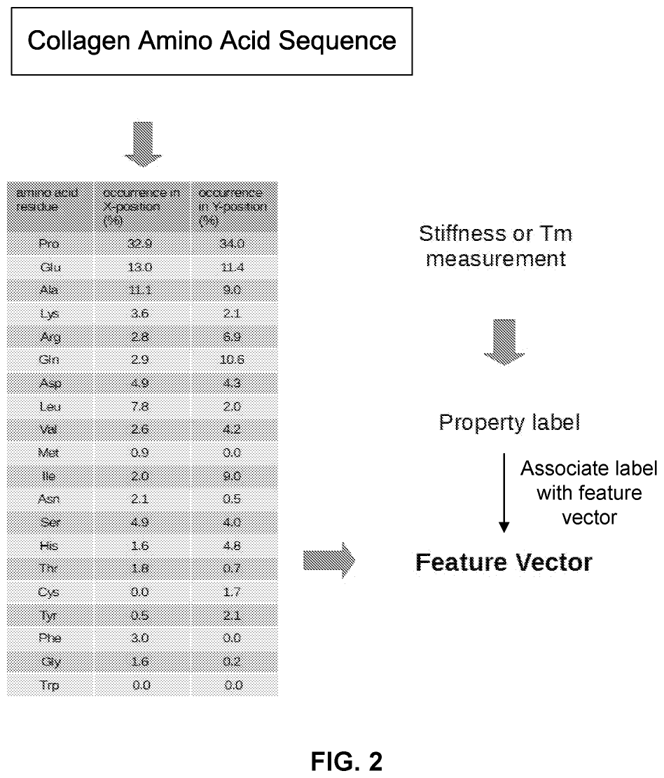

The collagen sequence data may be organized into frequencies of the amino acids. FIG. 2 illustrates how a feature vector may be generated and labeled by the physical properties of collagen or gelatin molecules or materials derived from the molecules. Generally for machine learning, a feature vector is an n-dimensional vector of numerical features that represent some object. The feature vector thus represents an observation of an object in an n-dimensional feature space. In some implementations as applied here, the features include amino acid information of collagen sequence as described below. An input feature vector to a supervised machine learning model can be labeled with a DV.

In some implementations, the sequences include the 20 standard amino acids as shown here. A collagen amino acid sequence is processed to provide frequencies of, e.g., 20 amino acid residues for the X position and the Y position of the X-Y-Gly trimer repeats of the collagen sequence, providing 40 frequencies (the number of amino acids times the number of positions considered). The 40 frequencies become 40 dimensions of the training data provided to the machine learning model. In this example, the frequencies are shown as the percentages of an amino acid relative to all possible amino acids at a particular position. Other forms of frequencies may be implemented, such as count and normalized counts of the amino acids. The values of frequencies of amino acids shown in the figure are for illustration purposes. They do not affect the implementations of the methods described herein.

FIG. 2 shows that the feature vector is associated with a property label indicating the physical or chemical property of a collagen-based material including collagen or gelatin molecules having the collagen sequence. In some implementations, the physical or chemical property is measured from a biomaterial derived from the molecule having the amino acid sequence. For example, the physical or chemical property can be stiffness or a melting temperature of the biomaterial derived from the collagen molecule.

In some implementations, the frequencies of amino acids indicate intra-sequence variation of amino acid trimers in a collagen sequence. In some implementations, such as in FIG. 2, the frequencies indicate how the X-Y-Gly trimers vary within the amino acid sequence. In some implementations, the frequencies of amino acids includes (a) a frequency for each of a plurality of different amino acids at the X positions of the X-Y-Gly trimers in each training sequence, and (b) a frequency for each of the plurality of different amino acids at the Y positions of the X-Y-Gly trimers in the training collagen sequence.

In some implementations, training a model includes removing amino acids that have low contribution to the physical or chemical properties based on the machine learning model, such as based on the weights or coefficients that the model associates with the amino acids. Therefore, after training, the amino acids provided to a model may include only a subset of the 20 standard amino acids and post-translationally modified amino acids of the subset.

In some implementations, the set of training data is generated using the main collagen domain with an uninterrupted X-Y-Gly trimer repeating sequence. For example, if a collagen sequence has the sequence of (Pro-Hyp-Gly)₁₀₀+(Pro-Glu-Gly)₅+(Pro-Hyp-Gly)₈, the (Pro-Hyp-Gly)₁₀₀ sequence is used as the training sequence.

In some implementations, the set of training data includes lengths of the plurality of training collagen sequences or lengths of fragments of the collagen sequences.

In some implementations, positional or regional information about the amino acid sequence is provided in the training set data. For example, in some implementations, an amino acid sequence can be divided into two or more regions. In some implementations, the amino acid sequence can be divided into three or more regions, including a C-terminal region, a middle region, and an N-terminal region. For example, if the sequence is divided into two regions, the frequencies of amino acids include the frequencies for the first region and the frequencies for the second region. More specifically, the frequencies of amino acids include: (a) a frequency for each of the plurality of different amino acids at X-positions of X-Y-Gly trimers in the first region of each training collagen sequence, (b) a frequency for each of the plurality of different amino acids at Y positions of X-Y-Gly trimers in the first region of each training collagen sequence, (c) a frequency for each of the plurality of different amino acids at the X-positions of the X-Y-Gly trimers in a second region of each training collagen or giant sequence, and (d) a frequency for each of the plurality of different amino acids at the Y positions of the X-Y-Gly trimers in the second region of each training collagen sequence. Similarly, the frequencies of amino acids can include frequencies for three or more regions of the amino acid sequence.

In some implementations, the at least one physical or chemical property includes one or more of the following: melting or gelling temperature, stiffness, elasticity, oxygen release rate, clarity, turbidity, ultraviolet blockage or absorption, viscosity, solubility, water content or hydration, resistance to protease, etc.

Physical or chemical properties can be measured using various methods reflecting various metrics such as Young's modulus, shear modulus, bulk modulus, etc. In some implementations, turbidity is measured by UV absorbance at 313 nm. Gelatin in solution, because of the high molecular weight of the protein, exists as a colloidal solution which scatters light, hence simple transmittance may not be a good measure for “clarity” for some conditions. In some implementations, the clarity of gelatin solutions can be measured using “nephelometry” in National Turbidity Units (NTU). In one example, it measures the a mount of light scattered from the light path at 90° as well as at 25° and compares it to the transmitted light beam, using a 4% solution of gelatine at 40° C. In other conditions, % transmittance at 640 nm can be used as a measure of clarity.

In some implementations, other optical properties of collagen or gelatin materials can be measured and modeled. For examples, direct measurements of melting temperature and heat effect of gelatin transitions from Differential Scanning calorimetry (DSC) can be modeled.

In some implementations, optical properties measured from fluorescent method can also be modeled. For instance, the method can model fluorescent depolarization, which requires the fluorescent dye, uranine (or other), to be absorbed by gelatin prior to the measurements. See, e.g. Hayashi and Oh, 1983, Agric. Biol. Chem.

In some implementations, the physical property can include viscosity, which is measured as the flow time of given volume of the solution through a standard pipette at constant temperature.

In the work flow, collagen or gelatin frequencies data are associated with at least one physical or chemical property. The association can be made as follows. In various implementations, a collagen sequence is processed to provide amino acid content information such as frequency data. The collagen sequence is comprised in a collagen or gelatin protein. A collagen protein can be transformed into gelatin by physical or chemical treatments. Biomaterials can be derived from the collagen or the gelatin. The collagen protein, the gelatin protein, and biomaterials derived from the collagen or gelatin each can have a physical or chemical property. The physical or chemical property can then be associated with the collagen sequence or the corresponding amino acid frequency data. In one sense, each type of collagen or gelatin molecule provides a single vector in a training set, and that vector includes (i) amino acid content information, and (ii) at least one chemical or physical property value.

In some implementations, two or more physical or chemical properties are provided in the training set data to train the model and to identify desirable collagen sequences.

As mentioned above, process 100 involves training the machine learning model by fitting the machine learning model to the set of training data. The type of machine learning model can be selected from any of the machine learning model types described hereinafter. In some implementations, the machine learning model is or includes a SVM model. In some implementations, the SVM has a linear kernel. In some implementations, the SVM has a nonlinear kernel. For a SVM having a linear kernel, some implementations further involves analyzing the weight vector of the SVM to determine which amino acids at which positions are the main determinants of the observed physical properties or chemical properties of the analyzed collagen samples. Then the feature space can be reduced by removing features (amino acids at specific position) that are unimportant in its contribution to the physical or chemical properties, which in effect reduces dimensionality of the feature space.

In some implementations, training the machine learning model involves applying a principal component analysis to the training data to reduce dimensionality of a feature space before providing the frequency data to train the machine learning model.

In some implementations, training a model includes using cross validation to select models that perform well. In cross validation, initially trained models are evaluated and compared. In some implementations, an amount (e.g., 10%) of training data is removed from the training set, machine learning models are retrained using the other 90% of vectors, and obtained models are tested on the remaining 10% validation set. This procedure could be repeated multiple times (e.g., 100 or more) by splitting the training and validation data repeatedly to avoid potential biases caused by the training set splitting. The results for models can be represented in a form of Receiver operating characteristic (ROC) and/or Precision-recall (PR) curve to evaluate the validity of the models.

In some implementations, linear SVM, non-linear SVM and random forests models can be compared using the cross-validation procedure described above. In some implementations, many models (of one type or multiple types) are generated. The models are compared based on their predictive abilities, and then one model or an ensemble of models can be selected. In some implementations, a genetic algorithm can be used to iteratively generate, select, and further refine models to develop models that are have high predictive power.

The best-performing method, as measured as the area under the ROC curve, is selected for further protein design. Obtaining the best-performing machine learning predictor allows for a rational design of recombinant collagens with desired physical-chemical properties (e.g., stiffness at the standard temperature or Tm).

In some implementations, the machine learning model includes a random forest model. In some implementations, the machine learning model includes a neural network model. In some implementations, the machine learning model includes a general linear model, such as a partial least squares model. Application of these model types to gelatin or collagen models is presented below.

Referring to FIG. 1, process 100 further involves obtaining, using the machine learning model, a set of target data predicted by the machine learning model to be associated with the at least one physical or chemical property meeting a criterion. See block 106. For example, the set of target data is predicted by the machine learning model to correspond to a gelatin that has a melting temperature above a criterion value, or has the highest clarity in a group.

Process 100 further involves determining one or more collagen sequences corresponding to the set of target data. See block 108. The target data includes frequencies of amino acids in the same way as the training data. So one set of amino acid frequency data can correspond to different collagen sequences. Other factors may be considered in identifying the collagen sequence corresponding to the set of target data. For example, in some implementations, the length of the collagen sequence is also processed by the machine learning model. So the length information may be combined with the frequency information to determine the collagen sequence. Also, in some implementations, the relative position information of the amino acids is processed by the machine learning model. Such positional or regional information can also be used to determine the collagen sequence to be produced. In some implementations, multiple collagen sequences are determined for one set of frequency data, and multiple collagen molecules can be produced.

Process 100 further involves producing one or more polynucleotides encoding the one or more collagen sequences. See block 110. In some implementations, the one or more polynucleotides include recombinant polynucleotides, which have sequence fragments corresponding to wild-type collagen sequence or mutant collagen sequence naturally occurring in organisms. In some implementations, the recombinant polynucleotides include designed fragments that do not naturally occur in organisms, but are recombined by genetically engineered organisms that do not naturally occur. In some implementations, the recombinant polynucleotides may be generated using chemical syntheses.

In some implementations, the one or more polynucleotides include polynucleotides generated de novo using oligonucleotide synthesizers. In some implementations, the polynucleotides include designed sequences not found in natural organisms.

Process 100 further involves expressing the one or more polynucleotides to produce one or more collagen molecules including the one or more collagen sequences. See block 110. Various expression systems may be used. In some implementations, the process uses an expression system including switched Escherichia Coli bacteria described hereinafter. In some implementations, the collagen molecules also include an amino acid sequence of a secretion tag. In some implementations, the secretion tag includes one or more of the following protein sequences: DsbA, pelB, OmpA, TolB, MalE, lpp, TorA, and HylA. The secretion tag causes the bacteria to secrete the collagen into the periplasmic space.

In some implementations, the one or more collagen molecules include amino acid sequences of one or more of the following: a histidine tag, a green fluorescent protein, a protease cleavage site, a beta-lactamase protein, etc.

In some implementations, process 100 optionally involves evolving the collagen molecules by using collagen sequences produced in block 112 to produce new gelatin products to generate a new set of training data, which is then used to further train a new machine learning model and identify further improved collagen sequences. Generating the new set of training data involves screening the collagen molecules to determine the physical or chemical property of the molecules or gelatin materials made from the molecules. See arrow 114 having the dashed line, the dash line indicating the step being optional.

In some implementations, SVM or general linear model (e.g., PLM) weights can be used to identify amino acids that can be modified to generate further improved collagen proteins in an iterative directed evolution process. For example, amino acids having high impact on physical or chemical properties as reflected by the model weights can be targeted for mutation or recombination. The mutated or recombined proteins are produced and screened for desired properties. Some implementations use the mutated or recombined proteins to provide training data to further develop the machine learning models.

In some implementations, process 100 further involves manufacturing gelatin or other materials from the one or more collagen molecules produced in block 112.

### IV Machine Learning Models

Machine learning is a field of computer science that gives computers the ability to learn to solve problems without being explicitly provided the solution. Evolved from the study of pattern recognition and computational learning theory in artificial intelligence, machine learning explores algorithms that can learn from and make predictions on data—such algorithms overcome following strictly static program instructions by making data-driven predictions or decisions through training a model using training data. Machine learning models use machine learning techniques to model physical phenomena or relationship among variables in the phenomena. Machine learning models are fit to the training data in a training phase, so the model can account for or “learn” the relationship in the training data.

Machine learning is considered supervised learning if feedback regarding its validity is given to the model during training. For example, if a model predicts a DV based on an IV, supervise learning provides training data that include both the IV and the DV of observations. Machine learning is considered unsupervised learning if feedback regarding its validity is not provided to the model during training. For example, if a model predicts a DV, e.g., a classification, based on an IV, unsupervised learning provides training data that include the IV but not DV of observations.

Some implementations disclosed herein provide a machine learning model for engineering collagen or gelatin proteins. The machine learning models receive, as input, frequency data of collagen or gelatin amino acid sequences. The machine learning models predict, or provide as output, values of one or more physical or chemical properties that are associated with the collagen or gelatin amino acid sequences. Therefore, the machine learning models can also be referred to as collagen frequency-property models.

In some embodiments, the machine learning model is a non-linear model. In other embodiments, it is a linear model. The machine learning models that may be used in the disclosed process include least squares models, partial least squares models, multiple linear regression, principal component regression, partial least squares regression, logistic regression, SVM, neural network, Bayesian linear regression, or bootstrap, and ensemble versions of these.

Linear Regression

Some implementations can use linear regression to model the relationship between collagen amino frequency and property. Linear regression provides a way of making quantitative predictions. In simple linear regression, a real-valued dependent variable (DV) Y is modeled as a linear function of a real-valued independent variable (IV) X plus noise:

Y=β0+β1X+ε

where β0 is an intercept, β1 a coefficient, and ε an error or deviation of data from the model.

In multiple regression, there are multiple independent variables X1, X2, . . . Xp≡X,

Y=β0+β^{T}X+ε

This works well when the effects of the IVs have strictly additive effects on Y, regardless of how the other variables behave. Otherwise, the model can be modified to account for interactions among IVs as follows.

Y=β0+β^{T}X+γXX^{T}+ε

Support Vector Machine Regression

Some implementations employ SVM regression to model the relation between collagen amino acid frequency and physical or chemical property. To illustrate, a simple example below describes a set of training data having only one IV (i.e., frequency of only one amino acid) and only one DV (e.g., melting temperature), each data point being (xᵢ,yᵢ). The SVM regression's goal is to find a function ƒ(x) that has at most ε deviation from the data yᵢ for all the training data, and at the same time is as flat as possible. In other words, the model does not care about errors as long as they are less than ε, but does not accept any deviation larger than ε.

In one form, a linear function is used as follows.

ƒ(x)= [symbol] w,x [symbol] +b

wherein [symbol] , [symbol] denotes a dot product. Flatness in the function above means that one seeks small w. Different measurements of “flatness” of the function may be used. One way to ensure this is to minimize the Euclidean norm of the function, ∥w∥². The solution is formalized as follows.

Minimize

$$\frac{1}{2} {‖ w ‖}^{2}$$

And satisfy

$$\{ \begin{matrix}{y}_{i} - 〈 w , {x}_{i} 〉 - b \leq ɛ \\ 〈 w , {x}_{i} 〉 + b - {y}_{i} \leq ɛ\end{matrix}$$

Euclidean norm of a vector is the magnitude of a vector. On an n-dimensional Euclidean space Rn, the intuitive notion of length of the vector x=(x1, x2, . . . , xn) is captured by the formula.

$$\frac{1}{2} {‖ X ‖}_{2} := \sqrt{{x}_{1}^{2} + … + {x}_{n}^{2}} .$$

In practice, it may not be possible to obtain the solution given actual data, because data points may fall outside of the error of ε. The model account for this using a soft margin to allow for further error. The model uses slack variables to relax the infeasible constraints of the optimization problem above. The problem is revised as.

Minimize

$$\frac{1}{2} {‖ w ‖}^{2} + C {\sum }_{i = 1}^{l} ( {ζ}_{i} + {ζ}_{i}^{*} )$$

And satisfy

$$\{ \begin{matrix}\begin{matrix}{y}_{i} - 〈 w , {x}_{i} 〉 - b \leq ɛ + {ζ}_{i} \\ 〈 w , {x}_{i} 〉 + b - {y}_{i} \leq ɛ + {ζ}_{i}^{*}\end{matrix} \\ {ζ}_{i} {ζ}_{i}^{*} \geq 0\end{matrix}$$

The constant C>0 determines the tradeoff between the flatness of ƒ(x) and the amount up to which deviations larger than c are tolerated.

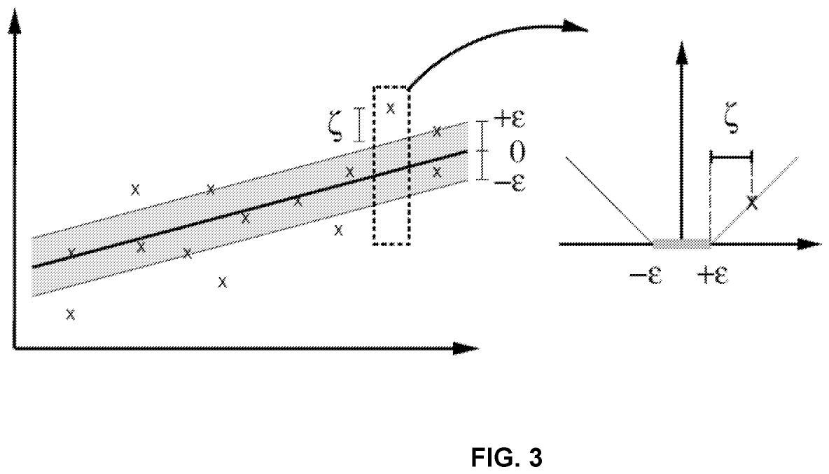

FIG. 3 graphically illustrates how the SVM regression models the data and finds the solution function. The subplot on the left shows the data points, the solution function, and the errors ε and ζᵢ. The subplot on the right shows the cost function. If the errors are within the shaded area corresponding to ε, it does not increase the cost. However, for errors beyond £, the cost increases linearly as shown on the right.

Random Forest

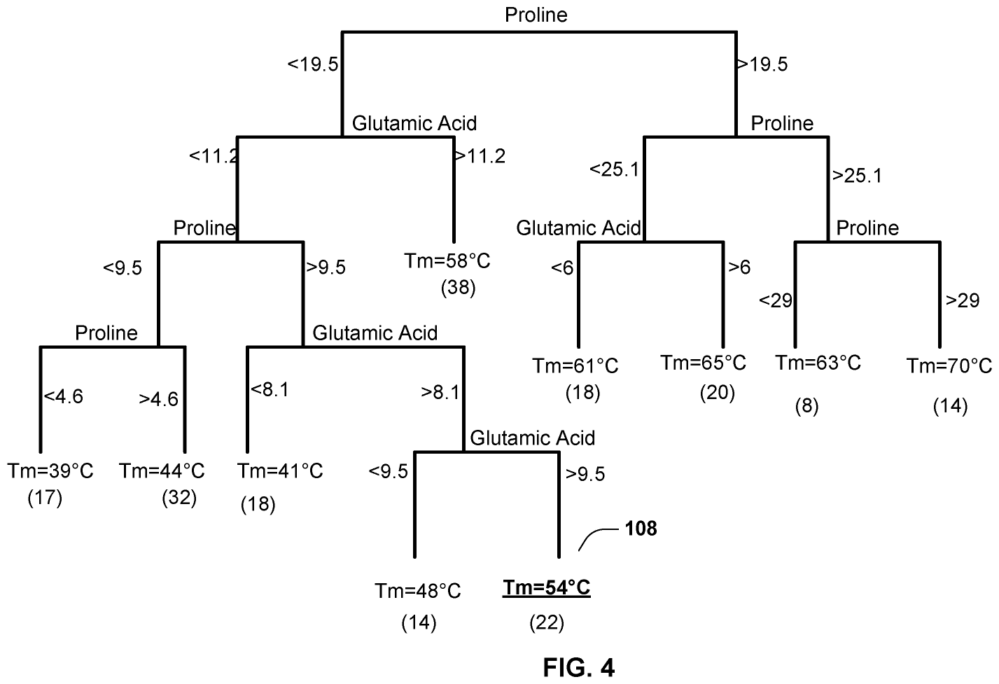

FIGS. 4-6 schematically illustrate how a random forests model can be built and applied to predict physical or chemical property of collagen molecules and materials derived therefrom.

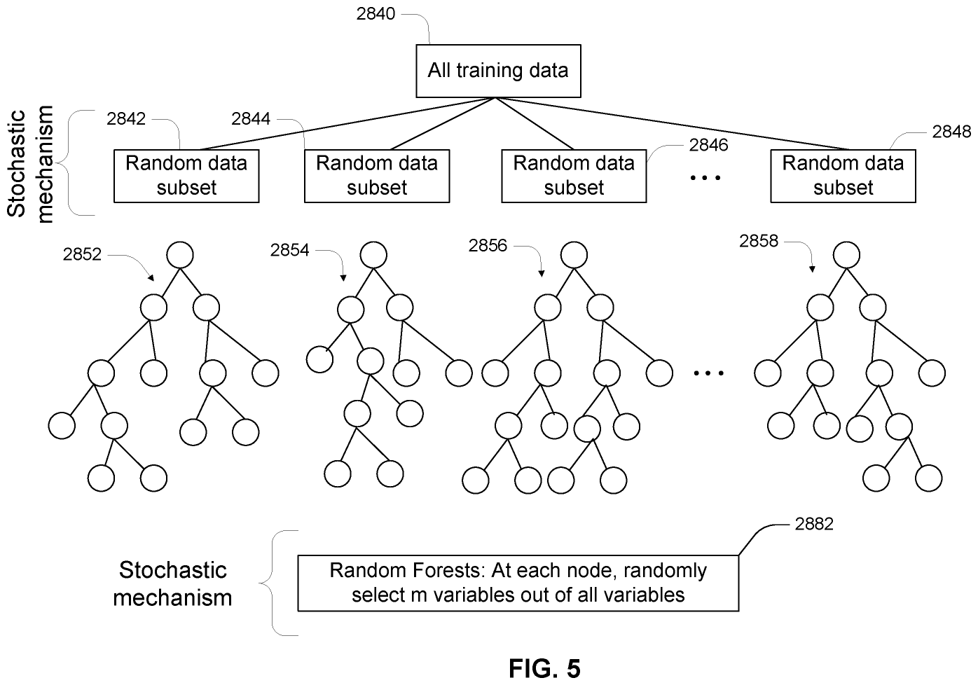

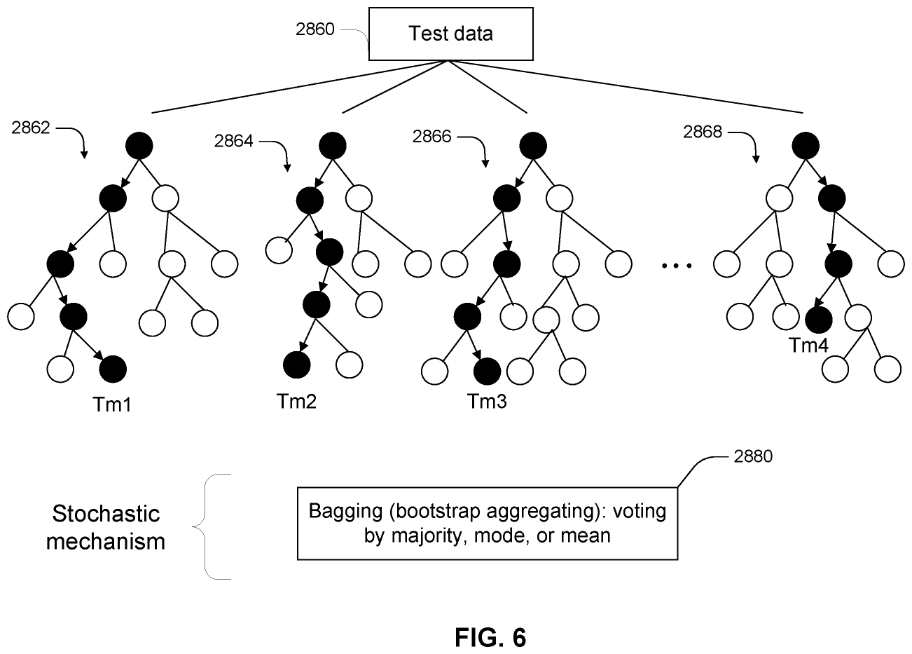

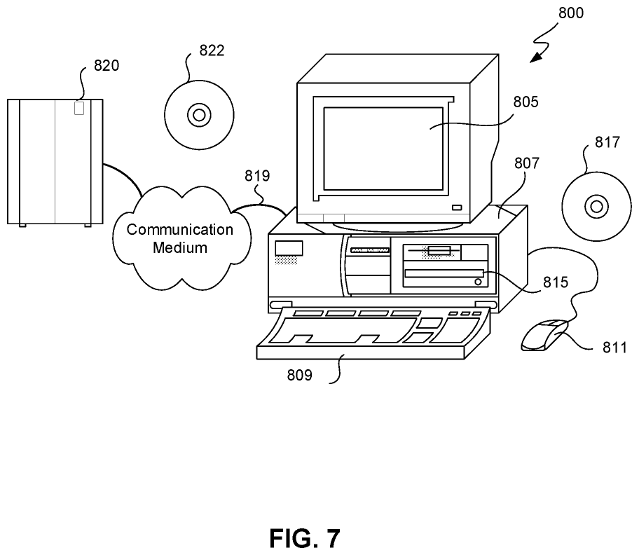

FIG. 4 shows a schematic, simplified decision tree for hypothetical data having only two dimensions—proline frequency and glutamic acid frequency in percentage. These decision trees are used to determine continuous values in a regression process, and are therefore also referred to regression trees. In this simplified illustrative example, each feature vector includes only two components: proline frequency and glutamic acid frequency in percentage. Each data point is labeled with a melting temperature (Tm). A training set of collagen molecules or collagen materials is used to train the decision tree. Once the decision tree is trained, testing data may be applied to the decision tree to predict the melting temperature of a test collagen. A number of decision trees are then combined with stochastic mechanisms as shown in FIGS. 5 and 6 to form a Random Forest.

The decision tree illustrated in FIG. 4 includes hypothetical data, which are for illustrative purpose only and do not reflect actual collagen sequences and their melting temperatures.

During a training phase, training collagen sequences are clustered in the two dimensional space, the clusters having different levels of melting temperature. Decision trees, such as the one shown in the FIG. 4, can be generated and trained to account for the clusters of the training collagen sequences. The decision tree in FIG. 4 has the number of training sequences at each leaf indicated by the numbers in the parentheses. The decision tree structure is formed such that its leaves correspond to the data points in clusters. During a test phase, the decision tree predicts a collagen sequence as follows. At a first decision node at the top (or root of the upside-down tree), it is checked whether it has a feature value of one or the other decision branch. If a data point belongs to one branch of a decision, it is then further determined which one of two branches at the next level it belongs to, until the data point is identified as belonging to an end node or a leaf of the decision tree. For example, a training collagen sequence has a proline frequency of 10% and a Glutamic acid frequency of 10%. The training sequence belongs to the left branch at the first level from the top, because its proline frequency of 10% is smaller than 19.5%. At the second level, it belongs to the left branch, because its glutamic acid frequency of 10% is smaller than 11.2%. At the third level, it belongs to the right branch, because its proline frequency of 10% is larger than 9.5%. At the fourth level, it belongs to the right branch, because its glutamic acid frequency of 10% is larger than 8.1%. At the fifth level, it belongs to the right branch, because its glutamic acid frequency of 10% is larger than 9.5%. So the decision tree predicts the collagen sequence to be associated with a melting temperature of 54° C.

FIGS. 5 and 6 illustrate using an ensemble of decision trees to perform regression including the stochastic mechanisms of the bootstrap aggregating (bagging) and Random Forest. In bagging, random data subset are selected from all available training data to train the decision trees. For example, a data subset 2842 is randomly selected with replacement from all training data 2840. The random data subset is also called a bootstrap data subset. The random data subset 2842 is then used to train the decision tree 2852. More random data subsets (2844-2848) are randomly selected as bootstrap data subsets and used to train decision trees 2854-2858.

In some implementations, the decision trees' predictive powers are evaluated using training data outside of the bootstrap data set. For instance, if a training data point is not selected in the data subset 2842, it can be used to test the predictive power of the decision tree 2852. Such testing is termed “out of the bag” or “oob” validation. In some implementations, decision trees having poor oob predictive power may be removed from the ensemble. Other methods such as cross-validation may also be used to remove low performing trees.

After the decision trees are trained and pruned, test data may be provided to the ensemble of decision trees to classify the test data. FIG. 28C illustrates how test data may be applied to an ensemble of decision trees to classify the test data 2860. For example, a test data point has one decision path in decision tree 2862 and is predicted to have Tm1. The same data point may be classified as Tm2 by decision tree 2864, as Tm3 by decision tree 2866, and Tm4 by decision tree 2868, and so on. Bagging method determines the final DV value by combining the results of all the individual decision trees. See block 2880. In classification applications, bagging can determine the final classification by voting by majority. It can also be determined as the mode of the classification distributions. In regression, bagging can determine the final classification by mean, mode, or median, weighted average, and other methods of combining outcomes from multiple trees.

Random Forest is further improves on bagging by integrating an additional stochastic mechanism into the ensemble of decision trees. In a Random Forest method, at each node of the decision tree, m variables are randomly selected from all of the available variables to train the decision node. See block 2882. It has been shown that the additional stochastic mechanism improve the accuracy and stability of the model.

### V. Collagen Expression System and Collagen Molecules

A number of protein expression systems can be used to express nucleic acid sequence obtained from the process disclosed above. In co-owned application PCT/US17/24857, incorporated by reference, an expression system that uses modified bacterial cells (switched cells) in which cell division is inhibited and growth of the periplasmic space is greatly enhanced was disclosed. In this expression system, the expressed proteins are targeted to the periplasmic space. Recombinant protein production in these switched cells is dramatically increased compared with that in non-switched cells. Structurally, the cells comprise both inner and outer membranes but lack a functional peptidoglycan cell wall, while the cell shape is spherical and increases in volume over time. Notably, while the periplasmic space normally comprises only 10-20% of the total cell volume, the periplasmic compartment of the switched state described herein can comprise more than 20%, 30%, 40% or 50% and up to 60%, 70%, 80% or 90% of the total cell volume.

The modified bacterial cells of PCT/US17/24857 are derived from Gram-negative bacteria, e.g. selected from: gammaproteobacteria and alphaproteobacteria. In some embodiments, the bacterium is selected from: Escherichia coli, Vibrio natriegens, Pseudomonas fluorescens, Caulobacter crescentus, Agrobacterium tumefaciens, and Brevundimonas diminuta. In specific embodiments, the bacterium is Escherichia coli, e.g. strain BL21(DE3).

In another aspect, the host bacterial cells have an enlarged periplasmic space in a culture medium comprising a magnesium salt, wherein the concentration of magnesium ions in the medium is at least about 3, 4, 5 or 6 mM. In further embodiments, the concentration of magnesium ions in the medium is at least about 7, 8, 9 or 10 mM. In some embodiments, the concentration of magnesium ions in the medium is between about 5 mM and 25 mM, between about 6 mM and/or about 20, 15 or 10 mM. In some embodiments, the magnesium salt is selected from: magnesium sulfate and magnesium chloride.

In other embodiments, the culture medium further comprises an osmotic stabilizer, including, e.g. sugars (e.g., arabinose, glucose, sucrose, glycerol, sorbitol, mannitol, fructose, galactose, saccharose, maltotrioseerythritol, ribitol, pentaerythritol, arabitol, galactitol, xylitol, iditol, maltotriose, and the like), betaines (e.g., trimethylglycine), proline, sodium chloride, wherein the concentration of the osmotic stabilizer in the medium is at least about 4%, 5%, 6%, or 7% (w/v). In further embodiments, the concentration of osmotic stabilizer is at least about 8%, 9%, or 10% (w/v). In some embodiments, the concentration of the osmotic stabilizer in the medium is between about 5% to about 20% (w/v).

In some embodiments, the cell culture medium further comprise ammonium chloride, ammonium sulfate, calcium chloride, amino acids, iron(II) sulfate, magnesium sulfate, peptone, potassium phosphate, sodium chloride, sodium phosphate, and yeast extract.

The host bacterial cell may be cultured continuously or discontinuously; in a batch process, a fed-batch process or a repeated fed-batch process.

In some embodiments, the cell culture medium further comprises one or more antibiotics. In some implementations, the antibiotic is selected from: β-lactam antibiotics (e.g. penicillins, cephalosporins, carbapenems, and monobactams), phosphonic acid antibiotics, polypeptide antibiotics, and glycopeptide antibiotics. In particular embodiments, the antibiotic is selected from alafosfalin, amoxicillin, ampicillin, aztreonam, bacitracin, carbenicillin, cefamandole, cefotaxime, cefsulodin, cephalothin, fosmidomycin, methicillin, nafcillin, oxacillin, penicillin g, penicillin v, fosfomycin, primaxin, and vancomycin.

Without being bound by theory, the cell morphology that promotes recombinant protein production and inhibits cell division appears to be driven by the removal of the cell wall under the media conditions stated above. In some embodiments, the methods for removal/inhibition of cell wall synthesis can be through the use of antibiotics that inhibit peptidoglycan synthesis (such as ampicillin, carbenicillin, penicillins or fosfomycin), or other methods known in the art.

When having an appropriate periplasmic targeting signal sequence, recombinantly produced polypeptides can be secreted into the periplasmic space of bacterial cells. Joly, J. C. and Laird, M. W., in The Periplasm ed. Ehrmann, M., ASM Press, Washington D.C., (2007) 345-360. The chemically oxidizing environment of the periplasm favors the formation of disulfide bonds and thereby the functionally correct folding of polypeptides.

In general, the signal sequence may be a component of the expression vector, or it may be a part of the exogenous gene that is inserted into the vector. The signal sequence selected should be one that is recognized and processed (i.e., cleaved by a signal peptidase) by the host cell. For bacterial host cells that do not recognize and process the native signal sequence of the exogenous gene, the signal sequence is substituted by any commonly known bacterial signal sequence. In some embodiments, recombinantly produced polypeptides can be targeted to the periplasmic space using the DsbA signal sequence. Dinh and Bernhardt, J Bacteriol, September 2011, 4984-4987. DsbA is a bacterial thiol disulfide oxidoreductase (TDOR). DsbA is a key component of the Dsb (disulfide bond) family of enzymes. DsbA catalyzes intrachain disulfide bond formation as peptides emerge into the cell's periplasm.

In some implementations, the non-naturally occurring collagen polypeptidefurther comprises amino acid sequences including a secretion tag. The secretion tag directs the collagen to the periplasmic space of the host cell. In particular embodiments, the signal peptide is derived from DsbA, pelB, OmpA, TolB, MalE, lpp, TorA, or HylA. In one aspect the secretion tag is attached to the non-naturally occurring collagen. In another aspect the secretion tag is cleaved from the non-naturally occurring collagen or elastin.

In some implementations, the non-naturally occurring collagen further comprises a histidine tag. The histidine tag or polyhistidine tag is a sequence of 2 to 20 histidine residues that are attached to the collagen. The histidine tag comprises 2 to 20 histidine residues, 5 to 15 histidine residues, 5 to 18 histidine residues, 5 to 16 histidine residues, 5 to 15 histidine residues, 5 to 14 histidine residues, 5 to 13 histidine residues, 5 to 12 histidine residues, 5 to 11, 5 to 10 histidine residues, 6 to 12 histidine residues, 6 to 11 histidine residues, or 7 to 10 histidine residues. The histidine tags are useful in purification of proteins by chromatographic methods utilizing nickel based chromatographic media. Exemplary fluorescent proteins include green fluorescent protein (GFP) or red fluorescent protein (RFP). Fluorescent proteins are well known in the art. In one embodiment the non-naturally occurring collagen comprises a GFP and/or RFP. In one embodiment a superfolder GFP is fused to the non-naturally occurring collagen. The superfolder GFP is a GFP that folds properly even when fused to a poorly folded polypeptide. In one aspect the histidine tag is attached to the non-naturally occurring collagen. In another aspect the histidine tag is cleaved from the non-naturally occurring collagen.

In some implementations, the non-naturally occurring collagen further comprises a protease cleavage site. The protease cleavage site is useful to cleave the recombinantly produced collagen to remove portions of the polypeptide. The portions of the polypeptide that may be removed include the secretion tag, the histidine tag, the fluorescent protein tag and/or the Beta-lactamase. The proteases comprise endoproteases, exoproteases serine proteases, cysteine proteases, threonine proteases, aspartic proteases, glutamic proteases, and metalloproteases. Exemplary protease cleavage sites include amino acids that are cleaved by Thrombin, TEV protease, Factor Xa, Enteropeptidase, and Rhinovirus 3C Protease. In one aspect the cleavage tag is attached to the non-naturally occurring collagen. In another aspect the cleavage tag is removed by an appropriate protease from the non-naturally occurring collagen.

In some implementations, the non-naturally occurring collagen further comprises an enzyme that is a Beta-lactamase. The beta-lactamase is useful as a selection marker. In one aspect the beta-lactamase is attached to the non-naturally occurring collagen or elastin. In another aspect the beta-lactamase is cleaved from the non-naturally occurring collagen or elastin.

The polynucleotides are in one aspect vectors used to transform host cells and express the polynucleotides. The polynucleotides further comprise nucleic acids that encode enzymes that permit the host organism to grow in the presence of a selection agent. The selection agents include certain sugars including galactose containing sugars or antibiotics including ampicillin, hygromycin, G418 and others. Enzymes that are used to confer resistance to the selection agent include β-galactosidase or a β-lactamase.

In one aspect the disclosure provides host cells that express the polynucleotides. Host cells can be any host cell including gram negative bacterial cells, gram positive bacterial cells, yeast cells, insect cells, mammalian cells, plant cells or any other cells used to express exogenous polynucleotides. An exemplary gram-negative host cell is E. coli.

The disclosure provides bacterial host cells in which the cells have been modified to inhibit cell division and the periplasmic space is increased. As discussed herein and taught in example 1, Beta-lactam antibiotics are useful as a switch to convert wild-type bacterial cells to a modified bacterial cell in which cell replication is inhibited and the periplasmic space is increased. Exemplary Beta-lactam antibiotics including penicillins, cephalosporins, carbapenems, and monobactams.

The switched form of bacteria (L-form) is cultivated in culture media that include certain salts and other nutrients. Salts and media compositions that support the physiological switch physiology that have been tested are M63 salt media, M9 salt media, PYE media, and Luria-Bertani (LB) media. Any necessary supplements besides carbon, nitrogen, and inorganic phosphate sources may also be included at appropriate concentrations introduced alone or as a mixture with another supplement or medium such as a complex nitrogen source. In certain embodiments, the medium further comprises one or more ingredients selected from: ammonium chloride, ammonium sulfate, calcium chloride, casamino acids, iron(II) sulfate, magnesium sulfate, peptone, potassium phosphate, sodium chloride, sodium phosphate, and yeast extract.

Beta-lactamases are enzymes that confer resistance to lactam antibiotics in prokaryotic cells. Typically when Beta-lactamases are expressed in bacterial host cells, the expressed Beta-lactamase protein also includes targeting sequences (secretion tag) that direct the Beta-lactamase protein to the periplasmic space. Beta-lactamases are not functional unless they are transported to the periplasmic space. This disclosure provides for targeting a Beta-lactamase to the periplasmic without the use of an independent secretion tag that targets the enzyme to the periplasmic space. By creating a fusion protein in which a periplasmic secretion tag added to the N-terminus of a protein such as GFP, collagen, or GFP/collagen chimeras, the functionality of the Beta-lactamase lacking a native secretion tag can be used to select for full translation and secretion of the N-terminal fusion proteins. Using this approach, we have used a DsbA-GFP-Collagen-Beta-lactamase fusion to select for truncation products in the target collagens that favor translation and secretion.

Another aspect provides a method of producing a non-naturally occurring collagen or a non-naturally occurring elastin. The method comprises the steps of inoculating a culture medium with a recombinant host cell comprising polynucleotides that encode the collagen, cultivating the host cell, and isolating the non-naturally occurring collagen or the non-naturally occurring elastin from the host cell.

The present disclosure furthermore provides a process for fermentative preparation of a protein, comprising the steps of:

a) culturing a recombinant Gram-negative bacterial cell in a medium comprising a magnesium salt, wherein the concentration of magnesium ions in the medium is at least about 6 mM, and wherein the bacterial cell comprises an exogenous gene encoding the protein;

b) adding an antibiotic to the medium, wherein the antibiotic inhibits peptidoglycan biogenesis in the bacterial cell; and

c) harvesting the protein from the medium.

The bacteria may be cultured continuously—as described, for example, in WO 05/021772—or discontinuously in a batch process (batch cultivation) or in a fed-batch or repeated fed-batch process for the purpose of producing the target protein. In some embodiments, protein production is conducted on a large-scale. Various large-scale fermentation procedures are available for production of recombinant proteins. Large-scale fermentations have at least 1,000 liters of capacity, preferably about 1,000 to 100,000 liters of capacity. These fermentors use agitator impellers to distribute oxygen and nutrients, especially glucose (the preferred carbon/energy source). Small-scale fermentation refers generally to fermentation in a fermentor that is no more than approximately 20 liters in volumetric capacity.

For accumulation of the target protein, the host cell is cultured under conditions sufficient for accumulation of the target protein. Such conditions include, e.g., temperature, nutrient, and cell-density conditions that permit protein expression and accumulation by the cell. Moreover, such conditions are those under which the cell can perform basic cellular functions of transcription, translation, and passage of proteins from one cellular compartment to another for the secreted proteins, as are known to those skilled in the art.

The bacterial cells are cultured at suitable temperatures. For E. coli growth, for example, the typical temperature ranges from about 20° C. to about 39° C. In one embodiment, the temperature is from about 25° C. to about 37° C. In another embodiment, the temperature is at about 30° C.

The pH of the culture medium may be any pH from about 5-9, depending mainly on the host organism. For E. coli, the pH is from about 6.8 to about 7.4, or about 7.0.

For induction of gene expression, typically the cells are cultured until a certain optical density is achieved, e.g., an OD600 of about 1.1, at which point induction is initiated (e.g., by addition of an inducer, by depletion of a repressor, suppressor, or medium component, etc.) to induce expression of the exogenous gene encoding the target protein. In some embodiments, expression of the exogenous gene is inducible by an inducer selected from, e.g. isopropyl-β-d-1-thiogalactopyranoside (IPTG), lactose, arabinose, maltose, tetracycline, anhydrotetracycline, vavlycin, xylose, copper, zinc, and the like.

After product accumulation, the cells are vortexed and centrifuged in order to induce lysis and release of recombinant proteins. The majority of the proteins are found in the supernatant but any remaining membrane bound proteins can be released using detergents (such as triton X-100).

In a subsequent step, the target protein, as a soluble or insoluble product released from the cellular matrix, is recovered in a manner that minimizes co-recovery of cellular debris with the product. The recovery may be done by any means, but in one embodiment, can comprise histidine tag purification through a nickel column. See, e.g., Purification of Proteins Using Polyhistidine Affinity Tags, Methods Enzymology. 2000; 326: 245-254.

In some implementations, a collagen polypeptide produced by the expression system includes an amino acid sequence of a secretion tag. In some implementations, the secretion tag includes one or more of the following: DsbA, pelB, OmpA, TolB, MalE, lpp, TorA, and HylA. In some implementations, the collagen polypeptide includes a plurality of X-Y-Gly trimers. Amino acids at X or Y positions of the X-Y-Gly trimers are selected from a group consisting of: alanine, cysteine, aspartic acid, glutamic acid, phenylalanine, glycine, histidine, isoleucine, lysine, leucine, methionine, asparagine, proline, pyrrolysine, glutamine, arginine, serine, threonine, selenocysteine, valine, tryptophan, tyrosine, and post-translational modifications therefrom. In some implementations, the collagen polypeptide is non-naturally occurring. The non-naturally occurring collagen polypeptide has been predicted by a machine learning model (such as the models described above) to be associated with at least one physical or chemical property meeting a criterion.

### VI. Digital Apparatus and Systems

As should be apparent, embodiments described herein employ processes acting under control of instructions and/or data stored in or transferred through one or more computer systems. Embodiments disclosed herein also relate to apparatus for performing these operations. In some embodiments, the apparatus is specially designed and/or constructed for the required purposes, or it may be a general-purpose computer selectively activated or reconfigured by a computer program and/or data structure stored in the computer. The processes provided by the present disclosure are not inherently related to any particular computer or other specific apparatus. In particular, various general-purpose machines find use with programs written in accordance with the teachings herein. However, in some embodiments, a specialized apparatus is constructed to perform the required method operations. One embodiment of a particular structure for a variety of these machines is described below.

In addition, certain embodiments of the present disclosure relate to computer readable media or computer program products that include program instructions and/or data (including data structures) for performing various computer-implemented operations. Examples of computer-readable media include, but are not limited to, magnetic media such as hard disks; optical media such as CD-ROM devices and holographic devices; magneto-optical media; and semiconductor memory devices such as flash memory and solid state drives (SSD). Hardware devices such as read-only memory devices (ROM) and random access memory devices (RAM) may be configured to store program instructions. Hardware devices such as application-specific integrated circuits (ASICs) and programmable logic devices (PLDs) may be configured to store program instructions and execute. It is not intended that the present disclosure be limited to any particular computer-readable media or any other computer program products that include instructions and/or data for performing computer-implemented operations.

Examples of program instructions include, but are not limited to low-level codes such as those produced by a compiler, and files containing higher level code that may be executed by the computer using an interpreter. Further, the program instructions include, but are not limited to machine code, source code and any other code that directly or indirectly controls operation of a computing machine in accordance with the present disclosure. The code may specify input, output, calculations, conditionals, branches, iterative loops, etc.

In one illustrative example, code embodying methods disclosed herein are embodied in a fixed media or transmissible program component containing logic instructions and/or data that when loaded into an appropriately configured computing device causes the device to perform a simulated genetic operation (GO) on one or more character string(s). FIG. 4 shows an example digital device 800 that is a logical apparatus that can read instructions from media 817, network port 819, user input keyboard 809, user input 811, or other inputting means. Apparatus 800 can thereafter use those instructions to direct statistical operations in data space, e.g., to construct one or more data set(s) (e.g., to determine a plurality of representative members of the data space). One type of logical apparatus that can embody disclosed embodiments is a computer system as in computer system 800 comprising CPU 807, optional user input devices keyboard 809, and GUI pointing device 811, as well as peripheral components such as disk drives 815 and monitor 805 (which displays GO modified character strings and provides for simplified selection of subsets of such character strings by a user. Fixed media 817 is optionally used to program the overall system and can include, e.g., a disk-type optical or magnetic media or other electronic memory storage element. Communication port 819 can be used to program the system and can represent any type of communication connection.

Certain embodiments can also be embodied within the circuitry of an application specific integrated circuit (ASIC) or programmable logic device (PLD). In such a case, the embodiments are implemented in a computer readable descriptor language that can be used to create an ASIC or PLD. Some embodiments of the present disclosure are implemented within the circuitry or logic processors of a variety of other digital apparatus, such as PDAs, laptop computer systems, displays, image editing equipment, etc.

In some embodiments, the present disclosure relates to a computer program product comprising one or more computer-readable storage media having stored thereon computer-executable instructions that, when executed by one or more processors of a computer system, cause the computer system to implement a method for engineering collagen. Such a method may be any method described herein such as those encompassed by the figures and pseudocode. In some embodiments, for example, the method includes (a) receiving a set of training data including frequencies of amino acid residues in a plurality of training collagen sequences and physical or chemical property data of the at least one physical or chemical property associated with the plurality of training collagen sequences; (b) training the machine learning model by fitting the machine learning model to the set of training data, wherein the trained machine learning model is configured to receive as input amino acid data of a test collagen sequence and predict at least one value for at least one physical or chemical property associated with the test collagen sequence. In some implementations, the method also includes (c) obtaining, using a machine learning model, a set of target data including frequencies of amino acid residues in one or more target collagen sequences, wherein the set of target data is predicted by the machine learning model to be associated with at least one physical or chemical property meeting a criterion; and (d) determining one or more collagen sequences corresponding to the set of target data.

In various embodiments, the computer system constructs a machine learning model by training a SVM model or other machine learning models. In various embodiments, the computer system uses the machine learning model to identify collagen sequences to form gelatin product with desired physical or chemical properties.

### VII. Examples

### Example 1: Expression System

Materials and methods:

Strains:

Tested Physiological Switch and Protein Production:

E. coli BL21(DE3)—From NEB, product #c2527

E. coli K12 NCM3722—From The Coli Genetic Stock Center, CGSC#12355

Tested Physiological Switch:

Gammaproteobacteria:

Vibrio natriegens—From ATCC, product #14048

Pseudomonas fluorescens—From ATCC, product #31948

Pseudomonas aeruginosa PAO1—From ATCC, product # BAA-47

Alphaproteobacteria:

Caulobacter crescentus—From ATCC, product #19089

Agrobacterium tumefaciens/Rhizobium radiobacter—From ATCC, product #33970

Brevundimonas diminuta—From ATCC, product #13184

Media Compositions:

1 Liter 5× m63 Salts:

10 g (NH4)₂SO₄—From P212121, product #7783-20-2

68 g KH₂PO₄—From P212121, product #7778-77-0

2.5 mg FeSO₄.7H2O—From Sigma Aldrich, product #F7002

Bring volume up to 1 liter with milliQ water

Adjust to pH 7 with KOH (From P212121, product #1310-58-3)

Autoclave mixture

1 Liter of 1M MgSO4:

246.5 g MgSO₄ 7 H2O—From P212121, (Sigma Aldrich, product #10034-99-8) Bring volume up to 1 liter with milliQ water.
Autoclave mixture.

1 Liter of Switch Media 1:

133.4 mL 5× m63 salts

### 10 mL 1M MgSO4

38.6 g Glucose—From P212121, product #50-99-7
66.6 g Sucrose—From P212121, product #57-50-1
8.33 g LB mix—From P212121, product #1b-miller
Bring volume up to 1 liter with milliQ water.
Filter sterilize mixture through a 0.22 μM pore vacuum filter (Sigma Aldrich, product #CLS430517).

1 Liter of Switch Media 2:

133.4 mL 5× m63 salts

### 10 mL 1M MgSO₄

38.6 g Glucose—From P212121, product #50-99-7
66.6 g Sucrose—From P212121, product #57-50-1
10 g Yeast Extract—From FisherSci.com, product #J60287A1
Bring volume up to 1 liter with milliQ water.
Filter sterilize mixture through a 0.22 μM pore vacuum filter (Sigma Aldrich, product #CLS430517).

For Bioreactor Growth:

5 liter of bioreactor media MGZ12:
1) Autoclave 1 L of Glucose at concentration of 500 g/L in DI water. (VWR, product #97061-170).
2) Autoclave 1 L of Sucrose at concentration of 500 g/L in DI water. (Geneseesci.com, product #62-112).
3) Autoclave in 3946 mL of DI water:
20 g (NH₄)₂HPO₄. (VWR, product #97061-932).
66.5 g KH₂PO₄. (VWR, product #97062-348).
22.5 g H₃C₆H₅O₇. (VWR, product #BDH9228-2.5 KG).
2.95 g MgSO₄.7H₂O. (VWR, product #97062-134).
10 mL Trace Metals (Teknova), 1000×. (Teknova, product #T1001).
After autoclaving add 400 mL of (1) to (3), 65 mL of 10M NaOH (VWR, product #97064-480) to (3), and 666 mL of (2) to (3).
A feed of 500 g/L of glucose can be used during fermentation run as needed.

At induction add:

50 mL of 1M MgSO₄.7H2O to a 5 L bioreactor
1 to 10 mM concentration of IPTG. (carbosynth.com, product # EI05931).
Add Fosfomycin (50 μg/mL or higher) and Carbenicillin (100 μg/mL or higher).

Physiological Switch:

The physiological switch is optimally flipped at an OD 600 of 1 to 1.1 for E. coli for growth in shake flasks at volumes up to 1 L. For the other species tested, cultures were grown in switch media and subcultured once cultures reached maximal OD 600. In all cases the physiological switch is flipped through the addition of 100-200 ug/mL Carbenicillin (From P212121, product #4800-94-6) and 50-100 ug/mL Fosfomycin (From P212121, product #26016-99-9). The majority of the population is in the switched state within a few hours. To confirm that cells underwent a physiological switch, cells were imaged on a Nikon Ti-E with perfect focus system, Nikon CFI60 Plan Apo 100× NA 1.45 objective, Prior automated filter wheels and stage, LED-CFP/YFP/mCherry and LED-DA/FT/TX filter sets (Semrock), a Lumencor Sola II SE LED illumination system, and a Hamamatsu Flash 4.0 V2 CMOS camera.

Image Analysis of Physiological Switch:

Images were analyzed using ImageJ to measure dimensions. In the switched state, the spherical outline of the outer membrane is treated as a sphere to calculate total volume (V=(4/3)πr3). The cytoplasmic volume is calculated as an ellipsoid that exists within the sphere (V=(4/3)π*(longest radius)*(short radius)2). To calculate the periplasmic volume, the cytoplasmic volume is subtracted from the total volume of the cell.

Protein Expression and Quantification:

E. coli BL21(DE3) (NEB product #c2527) containing pET28a (emd Millipore product #69864) and its derivatives carrying GFP or collagen derivatives were grown in a shaking incubator at 37° C. overnight in switch media containing 50 mg/mL kanamycin (p212121 product #2251180). Next day, subcultures are started with a 1:10 dilution of the overnight culture into fresh switch media containing 50 mg/mL kanamycin. The culture is then physiologically switched and protein production is induced simultaneously at an OD 600 of 1 to 1.1 (Read on a Molecular Devices Spectramax M2 microplate reader). The physiologically switch and protein production are flipped through the addition of 100 ug/mL Carbenicillin, 50 ug/mL Fosfomycin, and 100 ug/mL IPTG (p212121 product #367-93-1). Protein expression is continued in the switched state from between 8 hours to overnight at room temperature (approximately 22° C.) on an orbital shaker. In order to quantify total protein levels, Quick Start™ Bradford Protein Assay was used on mixed portion of culture and standard curves are quantitated on a Molecular Devices Spectramax M2 microplate reader. In order to quantitate the relative intensity of target protein production relative to the rest of the protein population the mixed portion of the cultures were run on Mini-PROTEAN® TGX™ Gels and stained with Bio-Safe™ Coomassie Stain.

Induction of Protein Production:

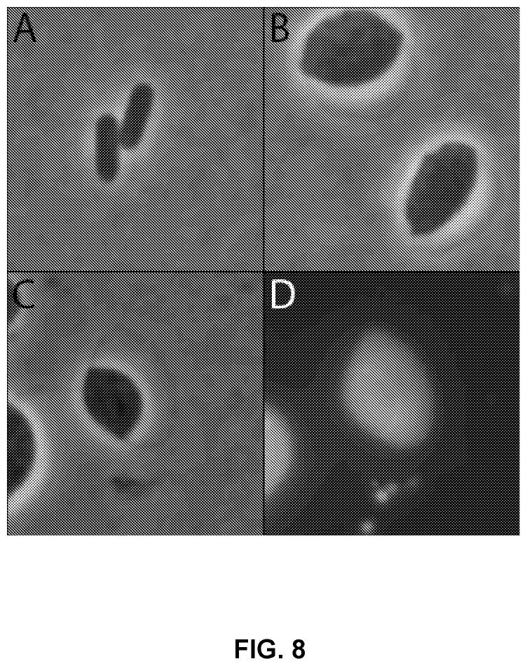

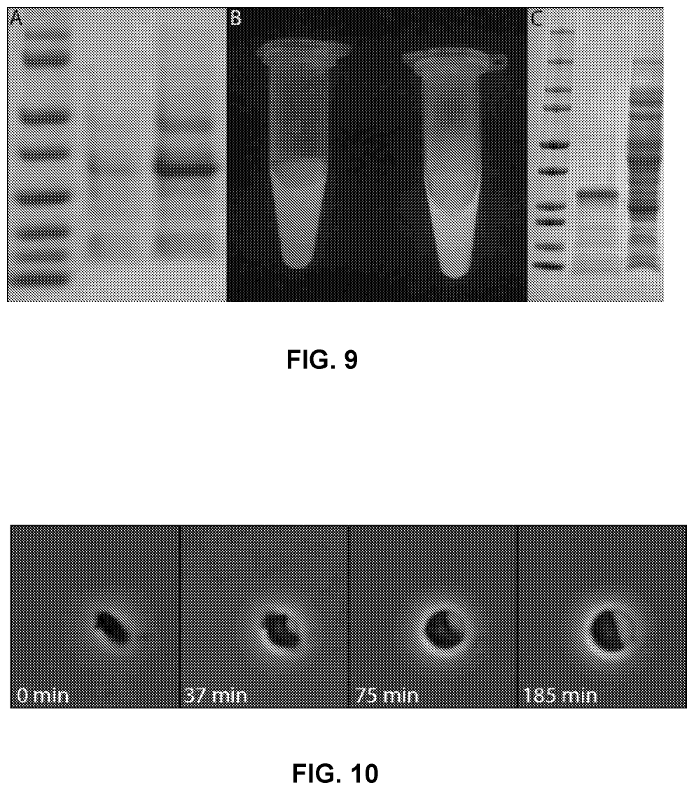

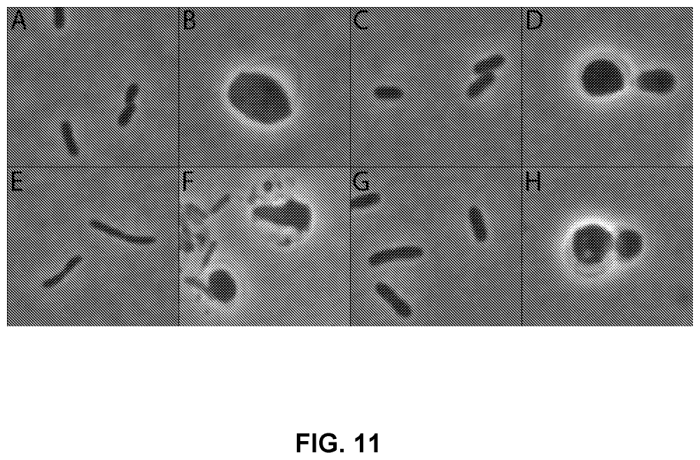

Standard procedures have been followed to induce protein production in the physiological state. We have been using the strain BL21(DE3) containing the plasmid pET28a driving the IPTG/lactose inducible production of recombinant proteins and targeting them to the periplasmic space using the DsbA signal sequence. Using the GFP protein, targeted to the periplasmic space as described above, we have demonstrated the ability to gain and increase of 5-fold in protein production when compared to un-switched cell populations induced at the same optical density, for the same amount of time (see FIGS. 8-11). The induction was optimal at an OD600 of 1.1 and induction was continued for 10 hours at which point the protein produced was measured at about 200 mg/mL.

### Example 2: Production of Collagen

Full length collagen can be produced using the method and system described herein. To illustrate the protein expression process, full length jellyfish collagen was produced using the expression system discussed in Example 1 herein. Similarly, collagen sequences obtained using machine learning models described above is manufactured and expressed. Collagens other than jelly fish collagen may also be produced using the same methodology.

In some implementations, truncated collagen sequences are expressed using the same method on the same system.

In some implementations, a set of target data comprising frequencies of amino acid residues in one or more target collagen sequences are obtained using a machine learning model as described above. The set of target data comprises frequencies of amino acid residues in one or more target collagen sequences. The set of target data has been predicted by the machine learning model to be associated with a physical or chemical property meeting a criterion. Then one or more collagen polypeptide sequences corresponding to the gelatin product with desired properties is obtained. In some implementations, a sequence can be a segment of the sequence of a molecule. The collagen polypeptide sequences can be full length or truncated sequences. Nucleic acids encoding a collagen polypeptide sequence are synthesized and expressed in a host cell. The expression of the polynucleotide is performed according to Example 1 or other known expression methodologies. In another embodiment the collagen polypeptide is directly synthesized using commercially available peptide synthesizers. The production of a full length jellyfish collagen using a polynucleotide is taught in this example.

The wild-type, full length amino acid sequence of Podocoryna carnea (jellyfish) collagen is provided in SEQ ID NO: 1.

(SEQ ID NO: 1)
GPQGVVGADGKDGTPGEKGEQGRTGAAGKQGSPGADGARGPLGSIGQQGA
RGEPGDPGSPGLRGDTGLAGVKGVAGPSGRPGQPGANGLPGVNGRGGLRG
KPGAKGIAGSDGEAGESGAPGQSGPTGPRGQRGPSGEDGNPGLQGLPGSD
GEPGEEGQPGRSGQPGQQGPRGSPGEVGPRGSKGPSGDRGDRGERGVPGQ
TGSAGNVGEDGEQGGKGVDGASGPSGALGARGPPGSRGDTGAVGPPGPTG
RSGLPGNAGQKGPSGEPGSPGKAGSAGEQGPPGKDGSNGEPGSPGKEGER
GLAGPPGPDGRRGETGSPGIAGALGKPGLEGPKGYPGLRGRDGTNGKRGE
QGETGPDGVRGIPGNDGQSGKPGIDGIDGTNGQPGEAGYQGGRGTRGQLG
ETGDVGQNGDRGAPGPDGSKGSAGRPGLR

https://www.ncbi.nlm.nih|.|gov/protein/4379341?report=genbank&log$=protalign&bl ast_rank=l&RID=T1N9ZEUW014

The non-codon optimized polynucleotide sequence encoding the full length jellyfish collagen is disclosed in SEQ ID NO: 2.

(SEQ ID NO: 2)
GGACCACAAGGTGTTGTAGGAGCTGATGGCAAAGATGGAACACCGGGAGA
GAAAGGTGAGCAAGGACGAACCGGAGCTGCAGGAAAACAGGGAAGCCCTG
GAGCAGATGGAGCAAGAGGCCCTCTTGGATCAATTGGACAACAAGGTGCT
CGTGGAGAACCTGGTGATCCAGGATCTCCCGGCTTAAGAGGAGATACTGG
ATTGGCTGGAGTCAAAGGAGTAGCAGGACCATCTGGTCGACCTGGACAAC
CCGGTGCAAATGGATTACCTGGTGTGAATGGCAGAGGCGGTTTGAGAGGC
AAACCTGGTGCTAAAGGAATTGCTGGCAGTGATGGAGAAGCGGGAGAATC
TGGCGCACCTGGACAGTCCGGACCTACCGGTCCACGTGGTCAACGAGGAC
CAAGTGGTGAGGATGGTAATCCTGGATTACAGGGATTGCCTGGTTCTGAT
GGAGAGCCCGGAGAGGAAGGACAACCTGGAAGATCTGGTCAACCAGGACA
GCAAGGACCACGTGGTTCCCCTGGAGAGGTAGGACCAAGAGGATCTAAAG
GTCCATCAGGAGATCGTGGTGACAGGGGAGAGAGAGGTGTTCCTGGACAA
ACAGGTTCGGCTGGAAATGTAGGAGAAGATGGAGAGCAAGGAGGCAAAGG
TGTCGATGGAGCGAGTGGACCAAGTGGAGCTCTTGGTGCTCGTGGTCCCC
CAGGAAGTAGAGGTGACACCGGGGCAGTGGGACCTCCCGGACCTACTGGG
CGATCTGGTTTACCTGGAAACGCAGGACAAAAGGGACCAAGTGGTGAACC
AGGTAGTCCAGGAAAAGCAGGATCAGCTGGTGAACAGGGTCCTCCTGGTA
AAGACGGATCAAATGGTGAACCTGGATCTCCTGGCAAAGAGGGTGAACGT
GGTCTTGCTGGTCCACCAGGTCCAGATGGCAGACGTGGTGAAACGGGATC
TCCAGGTATCGCTGGTGCTCTTGGTAAACCAGGTTTGGAAGGACCTAAAG
GTTATCCAGGATTAAGAGGAAGAGATGGAACCAATGGCAAACGAGGAGAA
CAAGGAGAAACTGGTCCTGATGGAGTCAGAGGTATTCCTGGAAATGATGG
ACAATCTGGCAAACCAGGTATTGATGGTATTGACGGAACAAATGGTCAAC
CAGGTGAGGCTGGATACCAAGGTGGTAGAGGTACACGTGGTCAGTTAGGT
GAAACTGGTGATGTCGGACAGAATGGAGATCGAGGAGCTCCTGGTCCTGA
TGGATCTAAAGGTTCTGCTGGTAGACCAGGACTTCGTGG

https://www.ncbi.nlm.nihllgov/nucleotide/3355656?report=genbank&log$=nuclalign &blast_rank=1&RID=TSYP7CMV014

Two different codon optimized polynucleotide sequences encoding the wild-type, full-length jellyfish collagen were synthesized. The two polynucleotide sequences were slightly different due to slightly different codon optimization methods. Polynucleotide sequences encoding other collagen sequences such as those determined using the machine learning model described above can be synthesized using the same method. In this example, in addition to the non-truncated, full-length jellyfish collagen, the polynucleotides also encoded a secretion tag, a 9 amino acid his tag, a short linker, and a thrombin cleavage site. The DsbA secretion tag is encoded by nucleotides 1-71. The histidine tag comprising 9 histidine residues is encoded by nucleotides 73-99 and encodes amino acids 25-33. The linker is encoded by nucleotides 100-111. The thrombin cleavage tag is encoded by nucleotides 112-135 and encodes amino acids 38-45. The truncated collagen is encoded by nucleotides 136-1422. The two polynucleotides are disclosed below in SEQ ID NO: 3 and 4.

(SEQ ID NO: 3)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGGGTCCGCAGGGTGTT
GTTGGTGCAGATGGTAAAGACGGTACCCCGGGTGAAAAAGGAGAACAGGG
ACGTACAGGTGCAGCAGGTAAACAGGGCAGCCCGGGTGCCGATGGTGCCC
GTGGCCCGCTGGGTAGCATTGGTCAGCAGGGTGCAAGAGGCGAACCGGGC
GATCCGGGTAGTCCGGGCCTGCGTGGTGATACGGGTCTGGCCGGTGTTAA
AGGCGTTGCAGGTCCTTCAGGTCGTCCAGGTCAACCGGGTGCAAATGGTC
TGCCGGGTGTTAATGGTCGTGGCGGTCTGCGTGGCAAACCGGGAGCAAAA
GGTATTGCAGGTAGCGATGGAGAAGCCGGTGAAAGCGGTGCCCCGGGTCA
GAGTGGTCCGACCGGTCCGCGCGGTCAGCGTGGTCCGTCTGGTGAAGATG
GCAATCCGGGTCTGCAGGGTCTGCCTGGTAGTGATGGCGAACCAGGTGAA
GAAGGTCAGCCGGGTCGTTCAGGCCAGCCGGGCCAGCAGGGCCCGCGTGG
TAGCCCGGGCGAAGTTGGCCCGCGGGGTAGTAAAGGTCCTAGTGGCGATC
GCGGTGATCGTGGTGAACGCGGTGTTCCTGGTCAGACCGGTAGCGCAGGT
AATGTTGGCGAAGATGGTGAACAGGGTGGCAAAGGTGTTGATGGTGCAAG
CGGTCCGAGCGGTGCACTGGGTGCACGTGGTCCTCCGGGCAGCCGTGGTG
ACACCGGTGCAGTTGGTCCGCCTGGCCCGACCGGCCGTAGTGGCTTACCG
GGTAATGCAGGTCAGAAAGGTCCGTCAGGTGAACCTGGCAGCCCTGGTAA
AGCAGGTAGTGCCGGTGAGCAGGGTCCGCCGGGCAAAGATGGTAGTAATG
GTGAGCCGGGTAGCCCTGGCAAAGAAGGTGAACGTGGTCTGGCAGGACCG
CCGGGTCCTGATGGTCGCCGCGGTGAAACGGGTTCACCGGGTATTGCCGG
TGCCCTGGGTAAACCAGGTCTGGAAGGTCCGAAAGGTTATCCTGGTCTGC
GCGGTCGTGATGGTACCAATGGCAAACGTGGCGAACAGGGCGAAACCGGT
CCAGATGGTGTTCGTGGTATTCCGGGTAACGATGGTCAGAGCGGTAAACC
GGGCATTGATGGTATTGATGGCACCAATGGTCAGCCTGGCGAAGCAGGTT
ATCAGGGTGGTCGCGGTACCCGTGGTCAGCTGGGTGAAACAGGTGATGTT
GGTCAGAATGGTGATCGCGGCGCACCGGGTCCGGATGGTAGCAAAGGTAG
CGCCGGTCGTCCGGGTTTACGTTAA

(SEQ ID NO: 4)
ATGAAAAAGATTTGGCTGGCGCTGGCTGGTTTAGTTTTAGCGTTTAGCGC
ATCGGCGGCGCAGTATGAAGATCACCATCACCACCACCACCATCACCACT
CTGGCTCGAGCCTGGTGCCGCGCGGCAGCCATATGGGTCCGCAGGGTGTT
GTTGGTGCAGATGGTAAAGACGGTACCCCGGGTGAAAAAGGTGAACAGGG
TCGTACCGGTGCAGCAGGTAAACAGGGCAGCCCGGGTGCCGATGGTGCCC
GTGGCCCGCTGGGTAGCATTGGTCAGCAGGGTGCACGTGGCGAACCGGGC
GATCCGGGTAGCCCGGGCCTGCGTGGTGATACGGGTCTGGCCGGTGTTAA
AGGCGTTGCAGGTCCTTCTGGTCGTCCAGGTCAACCGGGTGCAAATGGTC
TGCCGGGTGTTAATGGTCGTGGCGGTCTGCGTGGCAAACCGGGTGCAAAA
GGTATTGCAGGTAGCGATGGCGAAGCCGGTGAAAGCGGTGCCCCGGGTCA
GAGCGGTCCGACCGGTCCGCGCGGTCAGCGTGGTCCGTCTGGTGAAGATG
GCAATCCGGGTCTGCAGGGTCTGCCTGGTAGCGATGGCGAACCAGGTGAA
GAAGGTCAGCCGGGTCGTTCTGGCCAGCCGGGCCAGCAGGGCCCGCGTGG
TAGCCCGGGCGAAGTTGGCCCGCGCGGTTCTAAAGGTCCTAGCGGCGATC
GCGGTGATCGTGGTGAACGCGGTGTTCCTGGTCAGACCGGTAGCGCAGGT
AATGTTGGCGAAGATGGTGAACAGGGTGGCAAAGGTGTTGATGGTGCAAG
CGGTCCGAGCGGTGCACTGGGTGCACGTGGTCCTCCGGGCAGCCGTGGTG
ACACCGGTGCAGTTGGTCCGCCTGGCCCGACCGGCCGTAGCGGCCTGCCG
GGTAATGCAGGTCAGAAAGGTCCGTCTGGTGAACCTGGCAGCCCTGGTAA
AGCAGGTAGCGCCGGTGAGCAGGGTCCGCCGGGCAAAGATGGTAGCAATG
GTGAGCCGGGTAGCCCTGGCAAAGAAGGTGAACGTGGTCTGGCAGGTCCG
CCGGGTCCTGATGGTCGCCGCGGTGAAACGGGTTCTCCGGGTATTGCCGG
TGCCCTGGGTAAACCAGGTCTGGAAGGTCCGAAAGGTTATCCTGGTCTGC
GCGGTCGTGATGGTACCAATGGCAAACGTGGCGAACAGGGCGAAACCGGT
CCAGATGGTGTTCGTGGTATTCCGGGTAACGATGGTCAGAGCGGTAAACC
GGGCATTGATGGTATTGATGGCACCAATGGTCAGCCTGGCGAAGCAGGTT
ATCAGGGTGGTCGCGGTACCCGTGGTCAGCTGGGTGAAACCGGTGATGTT
GGTCAGAATGGTGATCGCGGCGCACCGGGTCCGGATGGTAGCAAAGGTAG
CGCCGGTCGTCCGGGTCTGCGTTAA

The amino acid sequence encoded by the polynucleotides of SEQ ID NO: 3 and SEQ ID NO:4 is disclosed in SEQ ID NO:5 below. The DsbA secretion tag is encoded by nucleotides 1-72 of SEQ ID NO: 3 or SEQ ID NO: 4, which encodes amino acids 1-24 of SEQ ID NO: 5; the histidine tag comprising 9 histidine residues is encoded by nucleotides 73-99 and encodes amino acids 25-33; the linker is encoded by nucleotides 100-111 and encodes amino acids 34-37; the thrombin cleavage tag is encoded by nucleotides 112-135 and encodes amino acids 38-45; the full-length collagen is encoded by nucleotides 136-1422 and encodes amino acids 46-474.

(SEQ ID NO: 5)
MKKIWLALAGLVLAFSASAAQYEDHHHHHHHHHSGSSLVPRGSHMGPQGV
VGADGKDGTPGEKGEQGRTGAAGKQGSPGADGARGPLGSIGQQGARGEPG
DPGSPGLRGDTGLAGVKGVAGPSGRPGQPGANGLPGVNGRGGLRGKPGAK
GIAGSDGEAGESGAPGQSGPTGPRGQRGPSGEDGNPGLQGLPGSDGEPGE
EGQPGRSGQPGQQGPRGSPGEVGPRGSKGPSGDRGDRGERGVPGQTGSAG
NVGEDGEQGGKGVDGASGPSGALGARGPPGSRGDTGAVGPPGPTGRSGLP
GNAGQKGPSGEPGSPGKAGSAGEQGPPGKDGSNGEPGSPGKEGERGLAGP
PGPDGRRGETGSPGIAGALGKPGLEGPKGYPGLRGRDGTNGKRGEQGETG
PDGVRGIPGNDGQSGKPGIDGIDGTNGQPGEAGYQGGRGTRGQLGETGDV
GQNGDRGAPGPDGSKGSAGRPGLR

The polynucleotides of SEQ ID NO: 3 and SEQ ID NO: 4 were synthesized by Gen9 DNA, now Gingko Bioworks internal synthesis. Overlaps between the pET28 vector and SEQ ID NO: 3 and SEQ ID NO: 4 were designed to be between 30 and 40 bp long and added using PCR with the enzyme PrimeStar GXL polymerase (http://www.clontech|.|com/US/Products/PCR/GC_Rich/PrimeSTAR_GXL_DNA_Po lymerase?sitex=10020:22372:US). The opened pET28a vector and insert DNA (SEQ ID NO: 3 or SEQ ID NO: 4) were then assembled together into the final plasmid using SGI Gibson assembly (https://us.vwr|.|com/store/product/17613857/gibson-assembly-hifi-1-step-kit-synthetic-genomics-inc). Sequence of plasmid was then verified through Sanger sequencing through Eurofins Genomics (www.eurofinsgenomics|.|com).

The transformed cells were cultivated in minimal media and frozen in 1.5 aliquots with glycerol at a ratio of 50:50 of cells to glycerol. One vial of this frozen culture was revived in 50 ml of minimal media overnight at 37° C., 200 rpm. Cells were transferred into 300 ml of minimal media and grown for 6-9 hours to reach an OD600 of 5-10.

Minimal media used in this example and throughout this application is prepared as follows. The minimal media (Table 1) was autoclaved in several separate fractions, Salts mix (Ammonium Phosphate dibasic, Potassium phosphate monobasic, Citric acid anhydrous, Magnesium sulfate heptahydrate), the Sucrose at 500 g/L, the Glucose at 55%, the Trace Metals TM5 (table 2), and Sodium Hydroxide 10M. The minimal media was then mixed together at the above concentrations post-autoclaving in the hood.

TABLE 1
Minimal media recipe for shake flask cultures

| chemical | Formula | MW | Conc (g/L) |
|---|---|---|---|
| Ammonium Phosphate dibasic | (NH₄)₂HPO₄ | 133 | 4 |
| Potassium phosphate monobasic | KH₂PO₄ | 137 | 13.3 |
| Citric acid anhydrous | H₃C₆H₅O₇ | 192.14 | 4.5 |
| Magnesium sulfate heptahydrate | MgSO₄•7H₂O | 246 | 0.59 |
| Trace Metals TM5 | | | 2 |
| Glucose | C₆H₁₂O₆ | 500 | 40 |
| Sodium Hydroxide 10M | NaOH | 400 | 5.2 |
| Sucrose 500 g/L | C₁₂H₂₂O₁₁ | 500 | 66.6 |

TABLE 2
Trace Metals TM5 composition

| chemical | Formula | MW | Conc (g/L) |
|---|---|---|---|
| Ferrous Sulfate Heptahydrate | FeSO₄•7H₂0 | 278.02 | 27.8 |
| Calcium Chloride | CaC₁₂•2H₂0 | 147 | 2.94 |
| Manganese Chloride | MnC₁₂ | 125.84 | 1.26 |
| Zinc Sulfate | ZnSO₄•H₂0 | 179.5 | 1.8 |
| Nickel Chloride | NiC₁₂•6H₂0 | 237.69 | 0.48 |
| Sodium Molybate | Na₂MoO₄•2H₂0 | 241.95 | 0.48 |
| Sodium Selenite | Na₂SeO₃ | 172.94 | 0.35 |
| Boric Acid | H₃BO₃ | 61.83 | 0.12 |

The harvested cells were disrupted in a homogenizer at 14,000 psi pressure in 2 passes. Resulting slurry contained the collagen protein along with other proteins.

The collagen was purified by acid treatment of homogenized cell broth. The pH of the homogenized slurry was decreased to 3 using 6M Hydrochloric acid. Acidified cell slurry was incubated overnight at 4° C. with mixing, followed by centrifugation. Supernatant of the acidified slurry was tested on a polyacrylamide gel and found to contain collagen in relatively high abundance compared to starting pellet. The collagen slurry thus obtained was high in salts. To obtain volume and salt reduction, concentration and diafiltration steps were performed using an EMD Millipore Tangential Flow Filtration system with ultrafiltration cassettes of 0.1 m² each. Total area of filtration was 0.2 m² using 2 cassettes in parallel. A volume reduction of 5× and a salt reduction of 19× was achieved in the TFF stage. Final collagen slurry was run on an SDS-PAGE gel to confirm presence of the collagen. This slurry was dried using a multi-tray lyophilizer over 3 days to obtain a white, fluffy collagen powder.

The purified collagen was analyzed on an SDS-PAGE gel and a thick and clear band was observed at the expected size of 42 kilodaltons. The purified collagen was also analyzed by mass spectrometry and it was confirmed that the 42 kilodalton protein was jellyfish collagen.

1. A method of engineering one or more collagen molecules comprising:
(a) obtaining, using a machine learning model and by a computer system comprising one or more processors and system memory, a set of target data comprising frequencies of amino acid residues in one or more target collagen sequences, wherein the set of target data is predicted by the machine learning model to be associated with at least one physical or chemical property meeting a criterion, wherein the machine learning model was obtained by:
(i) receiving a set of training data comprising frequencies of amino acid residues in a plurality of training collagen sequences and physical or chemical property data of the at least one physical or chemical property associated with the plurality of training collagen sequences; and
(ii) training the machine learning model by fitting the machine learning model to the set of training data, wherein the trained machine learning model is configured to receive as input amino acid data of a test collagen sequence and predict at least one value of the at least one physical or chemical property associated with the test collagen sequence;
(b) determining, by the computer system, one or more collagen sequences corresponding to the set of target data;
(c) producing one or more polynucleotides encoding the one or more collagen sequences; and
(d) expressing, on a protein production platform, the one or more polynucleotides to produce one or more collagen molecules comprising the one or more collagen sequences.

2. The method of claim 1, wherein the frequencies of amino acid residues indicates intra-sequence variation of amino acid trimers in the plurality of collagen sequences.

3. The method of claim 2, wherein the frequencies of amino acid residues comprise: (a) a frequency for each of a plurality of different amino acids as residues at X positions of X-Y-Gly trimers in each training collagen sequence, and (b) a frequency for each of the different plurality of amino acids as residues at Y positions of the X-Y-Gly trimers in the training collagen sequence.

4. The method of claim 3, wherein the plurality of different amino acids comprises 20 standard amino acids naturally occurring in organisms.

5. The method of claim 4, wherein the plurality of amino acids further comprises post-translational modifications of the 20 standard amino acids.

6. The method of claim 3, wherein the plurality of amino acids consists of a subset of 20 standard amino acids and post-translationally modified amino acids of the subset.

7. The method of claim 1, wherein the set of training data is generated using a main collagen domain with an uninterrupted (X-Y-Gly)ₙ repeating sequence.

8. The method of any of claim 1, wherein the set of training data comprises lengths of the plurality of training collagen sequences or fragments thereof.

9. The method of any of claim 1, wherein the frequencies of amino acid residues comprise: frequencies of amino acid residues in two or more regions of each training collagen sequence.

10. The method of any of claim 9, wherein the frequencies of amino acid residues comprise: (a) a frequency for each of a plurality of different amino acids at X positions of X-Y-Gly trimers in a first region of each training collagen sequence, (b) a frequency for each of a plurality of different amino acids at Y positions of X-Y-Gly trimers in the first region of each training collagen sequence, (c) a frequency for each of the plurality of different amino acids at the X positions of the X-Y-Gly trimers in a second region of each training collagen sequence, and (d) a frequency for each of the plurality of different amino acids at the Y positions of the X-Y-Gly trimers in the second region of each training collagen sequence.

11. The method of claim 1, wherein the machine learning model comprises a support vector machine.

12-13. (canceled)

14. The method of claim 11, wherein training the machine learning model comprises applying a linear support vector machine and a weight vector analysis to reduce dimensionality of a feature space.

15. The method of claim 1, wherein training the machine learning model comprises applying a principal component analysis to reduce dimensionality of feature space.

16. The method of claim 1, wherein the machine learning model comprises a random forest model, a neural network model, or a general linear model.

17-20. (canceled)

21. The method of claim 1, wherein the at least one physical or chemical property is selected from a group consisting of: melting or gelling temperature, stiffness, elasticity, oxygen release rate, clarity, turbidity, ultraviolet blockage or absorption, viscosity, solubility, water content or hydration, resistance to protease, and ability to associate into fibrils.

22. (canceled)

23. The method of claim 1, wherein the one or more polynucleotides comprise recombinant or synthesized polynucleotides.

24. (canceled)

25. The method of claim 1, wherein the one or more collagen molecules produced in (d) comprise recombinant collagen molecules.

26. The method of claim 1, further comprising manufacturing, using the one or more collagen molecules produced in (e), gelatin materials or collagen derivatives.

27. A non-naturally occurring collagen polypeptide comprising:
(a) an amino acid sequence of a secretion tag selected from the group consisting of DsbA, pelB, OmpA, TolB, MalE, lpp, TorA, and HylA; and
(b) a plurality of X-Y-Gly trimers,
wherein
(i) amino acids at X positions of the X-Y-Gly trimers are selected from a group consisting of: alanine, cysteine, aspartic acid, glutamic acid, phenylalanine, glycine, histidine, isoleucine, lysine, leucine, methionine, asparagine, proline, pyrrolysine, glutamine, arginine, serine, threonine, selenocysteine, valine, tryptophan, tyrosine, and post-translational modifications therefrom,
(ii) amino acids at Y positions of the X-Y-Gly trimers are selected from a group consisting of: alanine, cysteine, aspartic acid, glutamic acid, phenylalanine, glycine, histidine, isoleucine, lysine, leucine, methionine, asparagine, proline, pyrrolysine, glutamine, arginine, serine, threonine, selenocysteine, valine, tryptophan, tyrosine, and post-translational modifications therefrom, and
(iii) the non-naturally occurring collagen polypeptide was predicted by a machine learning model to be associated with at least one physical or chemical property meeting a criterion.

28-43. (canceled)

44. A computer system, comprising:
one or more processors;
system memory; and
one or more computer-readable storage media having stored thereon computer-executable instructions that, when executed by the one or more processors, cause the computer system to implement a method for engineering one or more collagen molecules, the one or more processors being configured to:
receive a set of training data comprising frequencies of amino acid residues in a plurality of training collagen sequences and physical or chemical property data of at least one physical or chemical property associated with the plurality of training collagen sequences; and
train a machine learning model by fitting the machine learning model to the set of training data, wherein the trained machine learning model is configured to receive as input amino acid data of a test collagen sequence and predict at least one value of the at least one physical or chemical property associated with the test collagen sequence.

45. (canceled)
